(19) United States
(12) Patent Application Publication
(10) Pub. No.: US 2013/0266575 A1
(43) Pub. Date: Oct. 10, 2013

(54) OprF/I AGENTS AND USE THEREOF IN HOSPITALIZED AND OTHER PATIENTS

(71) Applicant: Intercell AG, Vienna (AU)

(72) Inventors: Christoph Klade, Wr. Neustadt (AT); Robert Schlegl, Siegenfeld (AT); Kerstin Westritschnig, Vienna (AT)

(21) Appl. No.: 13/795,448

(22) Filed: Mar. 12, 2013

Related U.S. Application Data
(63) Continuation-in-part of application No. PCT/EP11/54127, filed on Mar. 18, 2011.
(60) Provisional application No. 61/426,760, filed on Dec. 23, 2010.

(51) Int. Cl. A61K 39/104

(52) U.S. Cl. CPC A61K 39/104
USPC 424/139.1; 424/185.1; 424/190.1

(57) ABSTRACT

The present invention relates to a new use of a vaccine comprising a fusion protein that comprises the Pseudomonas aeruginosa outer membrane protein I (OprI or OMPI) which is fused with its amino terminal end to the carboxy-terminal end of a carboxy-terminal portion of the Pseudomonas aeruginosa outer membrane protein F (OprF or OMPF), as well as to a new use of a monoclonal or polyclonal antibody against this fusion protein or a pharmaceutical composition thereof.

### RELATED APPLICATIONS

This application is a continuation-in-part of international application PCT/EP2011/054127, filed Mar. 18, 2011, which was published under PCT Article 21(2) in English, and claims the benefit under 35 U.S.C. §119(e) of U.S. provisional application Ser. No. 61/426,760, filed Dec. 23, 2010, the disclosures of which are incorporated by reference herein in their entireties.

### FIELD OF THE INVENTION

The present invention relates to a new use of a vaccine comprising a fusion protein that comprises the Pseudomonas aeruginosa outer membrane protein I (herein also referred to as “OprI” or “OMPI”) which is fused with its amino terminal end to the carboxy-terminal end of a carboxy-terminal portion of the Pseudomonas aeruginosa outer membrane protein F (herein also referred to as “OprF” or “OMPF”), as well as to a new use of a monoclonal or polyclonal antibody against this fusion protein or a pharmaceutical composition thereof.

### BACKGROUND OF THE INVENTION

Nosocomial infections are infections that are a result of treatment in a hospital or a healthcare service unit. Infections are considered nosocomial if they first appear 48 hours or more after hospital admission or within 30 days after discharge. This type of infection is also known as a hospital-acquired infection (or, in generic terms, healthcare-associated infection). In the United States, the Centers for Disease Control and Prevention estimates that roughly 1.7 million hospital-associated infections, from all types of microorganism, including bacteria, combined, cause or contribute to 99,000 deaths each year. In Europe, where hospital surveys have been conducted, the category of Gram-negative infections is estimated to account for two-thirds of the 25,000 deaths each year. Nosocomial infections can cause severe pneumonia and infections of the urinary tract, bloodstream and other parts of the body. Many types are difficult to attack with antibiotics, and antibiotic resistance is spreading to Gram-negative bacteria that can infect people outside the hospital.

In Gram-negative bacteria, lipopolysaccharides (LPS) and outer-membrane proteins are the major antigenic parts of the bacterial envelope. LPS based vaccines have been extensively studied in the 1970s (Priebe G, Pier G. Vaccines for Pseudomonas aeruginosa 2003. New Bacterial vaccines, edited by Ellis R W, Brodeur B. 260-82). Parke Davis produced a vaccine Pseudogen from LPS of 7 different serogroups. Some activity was observed with Pseudogen in non-randomized trials in cancer and burn patients but not in cystic fibrosis (CF) and leukemia patients. Being LPS based Pseudogen was very toxic and therefore not registered (Priebe, supra). Using two different versions of recombinant fusion proteins of Opr's F and I, von Specht and colleagues have shown that active immunization can protect neutropenic mice and passive immunization can protect SCID mice, both against a challenge dose 1000-fold above the LD50 (von Specht B U, Knapp B, Muth G et al. Protection of immunocompromised mice against lethal Infection with Pseudomonas aeruginosa by active or passive immunization with recombinant Pseudomonas aeruginosa outer membrane protein F and Outer membrane protein I fusion proteins. Infect Immun 1995; 63(5):1855-1862; Knapp B, Hundt E, Lenz U et al. A recombinant fusion outer membrane protein for vaccination against Pseudomonas aeruginosa. Vaccine 1999; 17(13-14):1663-1666). Said fusion protein was then tested for safety and immunogenicity in healthy volunteers reaching high levels of specific serum antibodies. To achieve an enhanced mucosal immunogenicity in cystic fibrosis an emulgel formulation of said fusion protein was developed and tested for safety and immunogenicity in healthy volunteers and lung impaired patients. However, the serum antibody response was comparatively low. A systemic i.m. booster has enhanced serum antibody response as compared to solely mucosal vaccination schedule.

An outer membrane protein preparation composed of 4 different strains of Pseudomonas aeruginosa with a molecular weight range of 10-100 kDa was developed as a vaccine in Korea. The vaccine contained minimal amounts of polysaccharide and was tested in a double-blind, placebo-controlled trial in burn patients (Jang I I, Kim I S, Park W J, et al. Human immune response to a Pseudomonas aeruginosa outer membrane protein vaccine. Vaccine 1999; 17(2): 158-68). Antibody levels to the vaccine antigens rose by 2.3-fold in the placebo group (19 patients) and 4.9 fold in the vaccine group (76 patients) (Kim D K, Kim J J, Kim J H et al. Comparison of two immunization schedules for a Pseudomonas aeruginosa outer membrane proteins vaccine in burn patients. Vaccine 2001; 19(9-10):1274-83). Priebe and Pier criticized the study because the follow-up of patients in the trial was incomplete, analysis was not by intention-to-treat, and there were no data regarding clinical outcomes (9). A similar Opr vaccine was tested in Russia 10 years earlier (Stanislaysky E S, Balayan S S, Sergienko A I, et al. Clinico-immunological trials of Pseudomonas aeruginosa vaccine. Vaccine 1991; 9(7):491-4). Pseudomonas aeruginosa vaccine (PV) containing predominantly cell-wall protein protective antigens was tested for safety and immunogenicity by immunization of 119 volunteers. The PV vaccine was well tolerated. A high level of specific antibodies persisted for the 5-month period of observation. The antibody titers increased in 94-97% of volunteers and moreover in 45.6% the antibody titers (the number of ELISA units) increased 2.5-3-fold and more. Anti-Pseudomonas aeruginosa plasma was used for the treatment of 46 patients with severe forms of Pseudomonas aeruginosa infection (40 adults and six infants aged up to 2 years) and 87% of the patients recovered. There have been no follow-up studies with the PV vaccine after 1991.

Hospital-acquired infections are one of the major causes of death and serious illness worldwide, resulting in an annual cost burden of more than USD 20 billion in the developed world. In the United States and Europe about 6 million patients become infected annually resulting in 140,000 deaths per year. The incidence of nosocomial infections is steadily increasing due to increasing medical interventions and antibiotic resistance. Thus, minimizing risk of mortality through hospital acquired infections by e.g. vaccination of burn victims and fibrosis patients, ICU patients and ventilated ICU patients is and is expected to become even more so a major unmet medical need in said patients.

### SUMMARY OF THE INVENTION

In accordance with the present invention, it has now surprisingly been found that a vaccine comprising a fusion protein that comprises the Pseudomonas aeruginosa outer membrane protein I (OprI or OMPI) or fragment thereof which is fused with its amino terminal end to the carboxy-terminal end of a carboxy-terminal portion of the Pseudomonas aeruginosa outer membrane protein F (OprF or OMPF), and in particular a non-adjuvanted vaccine comprising the fusion protein of SEQ ID NO: 1, reduced the mortality rate in mechanically ventilated intensive care patients significantly over alum as placebo control.

Mechanically ventilated intensive care patients are at particular risk of acquiring severe and often life-threatening forms of Pseudomonas aeruginosa or other infections, such as Ventilator-Associated Pneumonia (VAP), sepsis or soft tissue infection. Such infections also may affect burn victims, severely burned victims, cancer and transplant patients who are immunosuppressed, and cystic fibrosis patients, Intensive Care Unit (ICU) patients or generally all hospitalized patients.

Surprisingly, it was found by the inventors that a non-adjuvanted vaccine comprising a fusion protein, wherein OprI is linked with its N-terminal end to a C-terminal portion (e.g. as defined below) of OprF (herein also referred to as the “OprF/I agent” or “OprF/I fusion protein”) reduces significantly mortality over an alum only treatment as placebo control in mechanically ventilated intensive care patients. Furthermore, the alum-adjuvanted vaccine comprising the above fusion protein also showed reduced mortality compared to placebo.

Thus, in accordance with the particular findings of the present invention, there is provided:
1.1 A method of reducing mortality in a ventilated intensive care unit patient such as mechanically ventilated intensive care unit patient comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.2 A method of reducing mortality in a cystic fibrosis patient comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.3 A method of reducing mortality in a burn victim such as a first, second or third degree burn victim, preferably in a third degree burn victim, comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.4 A method of reducing mortality in cancer or transplant patients who are immunosuppressed comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.5 A method of reducing mortality in a intensive care unit patient comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.6 A method of reducing mortality in a hospitalized patient comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.7 A method of reducing mortality in a patient admitted to the intensive care unit with the need for mechanical ventilation for more than 48 hours comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.8 A method of reducing mortality in a human or non-human animal who will be operated or who is planning to be operated comprising administering to said human or non-human animal an effective amount (such as a pharmaceutically effective amount) of a pharmaceutical composition comprising an OprF/I agent, e.g. SEQ ID NO: 1 and optionally a pharmaceutically acceptable excipient, preferably at least 2 weeks before the planned operation;
1.9 A method of reducing mortality in a human that is at risk to be admitted to the intensive care unit such as a human who is doing extreme sports (such as base jumping, bungee jumping, gliding, hang gliding, high wire, ski jumping, sky diving, sky surfing, sky flying, indoor climbing, adventure racing, aggressive inline skating, BMX, caving, extreme motocross, extreme skiing, freestyle skiing, land and ice yachting, mountain biking, mountain boarding, outdoor climbing, sandboarding, skateboarding, snowboarding, snowmobiling, speed biking, speed skiing, scootering, barefoot waterskiing, cliff diving, free-diving, jet skiing, open water swimming, powerboat racing, round the world yacht racing, scuba diving, snorkeling, speedsailing, surfing, wakeboarding, whitewater kayaking, windsurfing) comprising administering to said human an effective amount (such as a pharmaceutically effective amount) of a pharmaceutical composition comprising an OprF/I agent, e.g. SEQ ID NO: 1 and optionally a pharmaceutically acceptable excipient, preferably at least 2 weeks before the planned extreme sport event;
1.10 A method of reducing mortality in a human, in particular a human of any age, e.g. of age 2 or older, comprising administering to said human an effective amount (such as a pharmaceutically effective amount) of a pharmaceutical composition comprising an OprF/I agent, e.g. SEQ ID NO: 1 and optionally a pharmaceutically acceptable excipient, preferably wherein said method additionally comprises regular booster vaccinations;
1.11 A method of reducing mortality in a human with any kind of infection, comprising administering to said human an effective amount (such as a pharmaceutically effective amount) of a pharmaceutical composition comprising an OprF/I agent, e.g. SEQ ID NO: 1 and optionally a pharmaceutically acceptable excipient, preferably wherein said method additionally comprises regular booster vaccinations;
1.12 A method as defined above, wherein the OprF/I agent is selected from the group consisting of a polypeptide consisting of i) SEQ ID NO: 2, 3, 7 to 10, and ii) SEQ ID NO: 4; SEQ ID NO: 1 SEQ ID NO: 11 to 13; and an antibody or antigen-binding portion thereof directed against said polypeptide or SEQ ID NO: 1;
1.13 A method as defined above, wherein the pharmaceutical composition is a vaccine;
1.14 A method as defined above, wherein the pharmaceutical composition is a vaccine that is non-adjuvanted.
1.15 A method as defined above, comprising co-administration of a first drug substance, said first drug substance being an effective amount (such as a pharmaceutically effective amount) of a vaccine comprising an OprF/I agent, e.g. SEQ ID NO: 1, and a second drug substance, said second drug substance being an effective amount (such as a pharmaceutically effective amount) of an agent selected from the group consisting of antibiotics such as intravenous antibiotics and other drug substances improving the state of the patient, human, or non-human animal in particular in regards to reducing the risk of mortality;
1.16 A method as defined above, wherein the mortality is lower than 100.
1.17 A method as defined above, wherein the mortality e.g. in an ICU patient, preferably in ventilated ICU patients, is lower than 95, preferably 90, more preferably 85, more preferably 80, more preferably 75, more preferably 70, more preferably 65, even more preferably lower than 60, most preferably lower than 55.
1.18 A method as defined above, wherein the OprF/I agent is a protein complex comprising (or consisting at least 80%, preferably 85%, more preferably 90% of) three OprF/I agents with SEQ ID NO: 1 or an functional active variant thereof having at least 85%, preferably 90%, in particular 95% identity to the amino acid sequence of SEQ ID NO:1;
1.19 A method as defined above, wherein the OprF/I agent is selected from the group consisting of
   (a) the OprF/I agent of SEQ ID NO: 1 with a Cys18-Cys27-bond (SEQ ID NO: 11), and
   (b) the OprF/I agent of SEQ ID NO: 1 with a Cys18-Cys27-bond and Cys33-Cys47-bond (SEQ ID NO: 12), and
   (c) the OprF/I agent of SEQ ID NO: 1 with a Cys18-Cys47-bond and Cys27-Cys33-bond (SEQ ID NO: 13),
   or an functional active variant thereof having at least 85%, preferably 90%, in particular 95% identity to the amino acid sequence of SEQ ID NO: 1, and the same disulphide bond pattern as specified in (a), (b) or (c);
   preferably the OprF/I agent is a protein complex comprising three OprF/I agents with SEQ ID NO: 1 or a functional active variant thereof having at least 85%, preferably 90%, in particular 95% identity to the amino acid sequence of SEQ ID NO:1, and the sum of a) the OprF/I agent of SEQ ID NO: 1 with a Cys18-Cys27-bond (SEQ ID NO: 11), b) the OprF/I agent of SEQ ID NO: 1 with a Cys18-Cys27-bond and Cys33-Cys47-bond (SEQ ID NO: 12), and c) the OprF/I agent of SEQ ID NO: 1 with a Cys18-Cys47-bond and Cys27-Cys33-bond (SEQ ID NO: 13) is equal or greater than 75%.

Suitable second drug substances may include e.g. antibiotics such as i) an antimicrobial compound, e.g. penicillins, cephalosporins, polymixins, quinolones, sulfonamides, aminoglycosides, macrolides, tetracyclines, daptomycins, tigecyclines, linezolids; ii) an antifungal compound, e.g. polyene antimycotics, natamycin, rimocidin, filipin, nyastatin, amphotericin B, candicin, hamycin.

The terms “co-administration” or “combined administration” or the like as utilized herein are meant to encompass administration of the selected therapeutic or prophylactic agents to a single patient, and are intended to include treatment or prophylactic regimens in which the agents are not necessarily administered by the same route of administration or at the same time.

### BRIEF DESCRIPTION OF THE DRAWINGS

FIG. 1: Primary endpoints on immunogenicity met—Summary of immunogenicity of study results (GMT: Geometric Mean Titers)

FIG. 2: Reduction in mortality vs. placebo in all vaccine groups. Statistically significant reduction of mortality for group vaccinated with 100 mcg w/o Alum (p=0.0196 at Day 28)

FIG. 3: Significant prognostic value of OprF/I titers on survival. Anti-OprF/I (SEQ ID NO: 1) IgG TITERS was measured at day 14. Cox regression analysis demonstrated a significant prognostic value of the OprF/I IgG titer on survival (p=0.0336)

FIG. 4: Reduction in mortality vs placebo in patients with infections. Subgroup C (interrupted line): Patients with any investigator confirmed infection overall. Subgroup D (un-interrupted line): Patients without any investigator confirmed infection overall

FIG. 5 schematically depicts the reduction and controlled reoxidation processes according to the present invention.

FIG. 6 shows the superimposition of RP-HPLC profiles of the OprF/I fusion protein after expression and capturing on IMAC, after reduction, and after reoxidation/purification.

FIG. 7 shows the superimposition of SEC profiles of the OprF/I fusion protein after expression and capturing on IMAC, and after reoxidation/purification.

FIG. 8 shows the RP-HPLC analysis of the reoxidized IMAC/G50 pool. Samples were analyzed after 300 minutes and 21 hours.

FIG. 9 shows the change in retention time during SEC analysis of OprF/I fusion protein samples at pH 8.0 and pH 2.

FIG. 10 shows a flow scheme of an exemplary production and purification process of the OprF/I fusion protein.

FIG. 11 shows preparative and analytical RP-HPLC elution profiles of an elected QSHP fraction.

FIG. 12 shows the disulphide bond pattern of peaks P1, P2 and P3 of the OprF/I fusion protein.

### DETAILED DESCRIPTION OF THE INVENTION

### Definitions

Unless defined otherwise, all technical and scientific terms used herein have the same meaning as commonly understood by those of ordinary skill in the art to which this invention pertains.

The term “antibody” as used herein includes whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2. FR3. CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody, as used herein, refers to one or more fragments of an intact antibody that retain the ability to specifically bind to a given antigen (e.g., an OprF/I agent or SEQ ID NO: 1). Antigen binding functions of an antibody can be performed by fragments of an intact antibody. Examples of binding fragments encompassed within the term “antigen-binding portion” of an antibody include a Fab fragment, a monovalent fragment consisting of a camelized VH or dAb domain, a F(ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989 Nature 341:544-546) which consists of a VH domain or a VL domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 65:5879-5883). Such single chain antibodies include one or more “antigen-binding portions” of an antibody. These antibody fragments are obtained using conventional techniques known to those of skill in the art, and the fragments are screened for utility in the same manner as are intact antibodies. Antigen-binding portions can also be incorporated into single domain antibodies, maxibodies, minibodies, intrabodies, diabodies, triabodies, tetrabodies. v-NAR and bis-scFv (see, e.g., Hollinger and Hudson. 2005. Nature Biotechnology, 23, 9, 1126-1136). Antigen-binding portions of antibodies can be grafted into scaffolds based on polypeptides such as Fibronectin type III (Fn3) (see U.S. Pat. No. 6,703,199, which describes fibronectin polypeptide monobodies). Antigen-binding portions can be incorporated into single chain molecules comprising a pair of tandem Fv segments (VH-CHI-VH-CMI) which, together with complementary light chain polypeptides, form a pair of antigen binding regions (Zapata et al., 1995 Protein Eng. 8(10): 1057-1062; and U.S. Pat. No. 5,641,870).

As used herein, the term “Affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “Avidity” refers to an informative measure of the overall stability or strength of the antibody-antigen complex. It is controlled by three major factors: antibody epitope affinity; the valency of both the antigen and antibody; and the structural arrangement of the interacting parts. Ultimately these factors define the specificity of the antibody, that is the likelihood that the particular antibody is binding to a precise antigen epitope.

The term “amino acid” refers to naturally occurring and synthetic amino acids, as well as amino acid analogs and amino acid mimetics that function in a manner similar to the naturally occurring amino acids. Naturally occurring amino acids am those encoded by the genetic code, as well as those amino acids that are later modified, e.g., hydroxyproline, y-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g. norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “binding specificity” as used herein refers to the ability of an individual antibody combining site to react with only one antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody. Specific binding between two entities means a binding with an equilibrium constant (KA) of at least 1×10⁷ M⁻¹, 10⁸ M⁻¹, 10⁹ M⁻¹, 10¹⁰ M⁻¹, 10¹¹ M⁻¹, 10¹² M⁻¹, 10¹³ M⁻¹. The phrase “specifically (or selectively) binds” to an antibody (e.g., an OprF/I agent-binding antibody) refers to a binding reaction that is determinative of the presence of an antigen (e.g., an OprF/I agent) in e.g. a heterogeneous population of proteins and other compounds. In addition to the equilibrium constant (KA) noted above, an OprF/I agent-binding antibody of the invention typically also has a dissociation rate constant (KD) of about 10⁻⁸ M, 10⁻⁹ M, or 10⁻¹⁹ M, 10⁻¹¹ M, or 10⁻¹² M, or 10⁻¹³ M or lower, and binds to the OprF/I agent(s) with an affinity that is at least 10-fold, preferably 100-fold, or up to 1000-fold or more greater than its affinity for binding to a non-specific antigen. The phrases “an antibody recognizing an antigen” and “an antibody specific for an antigen” are used interchangeably herein with the term “an antibody which binds specifically to an antigen”.

The term “KA” or “Ka”, as used herein, is intended to refer to the association rate of a particular antibody-antigen interaction, whereas the term “KD” or “Kd”, as used herein, is intended to refer to the dissociation rate of a particular antibody-antigen interaction. These values can be determined using methods well established in the art, e.g., a surface plasmon resonance or a biosensor system such as a Biacore® system.

As used herein, the term “subject” includes any human or non-human animal.

The term “non-human animal” includes all nonhuman vertebrates, e.g. mammals and non-mammals, such as nonhuman primates, rodents, rabbits, sheep, dogs, cats, horses, cows, birds, amphibians, reptiles, etc.

The term “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g. an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations.” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see e.g. Creighton. Proteins (1984)). In some embodiments, the term “conservative sequence modifications” are used to refer to amino acid modifications that do not significantly affect or alter the binding characteristics of the antibody containing the amino acid sequence.

The term “functional active variant” means a sequence variant that exhibits the biological activity of the sequence from which it is derived (i.e., without the sequence alterations found in the variant), particularly, if the activity of the variant amounts to at least 10%, preferably at least 25%, more preferably at least 50%, even more preferably at least 70%, still more preferably at least 80%, especially at least 90%, particularly at least 95%, most preferably at least 99% of the activity of the sequence without sequence alterations.

In a preferred embodiment the term “functional active variant” means “immunogenic variant,” which is defined as a sequence variant which shows in vivo immunogenicity, e.g. in the BALB/c mouse model, e.g. have an ED50 value of 10 μg of lower, more preferably an ED50 value of 5 μg or lower such as e.g. 4 μg or lower, 3 μg or lower or 2 μg or lower (see Examples section).

The terms “cross-block”, “cross-blocked” and “cross-blocking” are used interchangeably herein to mean the ability of an antibody or other binding agent to interfere with the binding of other antibodies or binding agents to any of the OprF/I agents in a standard competitive binding assay.

The ability or extent to which an antibody or other binding agent is able to interfere with the binding of another antibody or binding molecule to an OprF/I agent, and therefore whether it can be said to cross-block according to the invention, can be determined using standard competition binding assays. One suitable assay involves the use of the Biacore technology (e.g. by using the BIAcore 3000 instrument (Biacore, Uppsala, Sweden)), which can measure the extent of interactions using surface plasmon resonance technology. Another assay for measuring cross-blocking uses an ELISA-based approach.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

As used herein, the term “high affinity” for an IgG antibody or fragment thereof (e.g., a Fab fragment) refers to an antibody having a KD of 10⁻⁸ M or less, 10⁻⁹ M or less, or 10⁻¹⁹ M, or 10⁻¹¹ M or less, or 10⁻¹² M or less, or 10⁻¹³ M or less for a target antigen. However, “high affinity” binding can vary for other antibody isotypes. For example, “high affinity” binding for an IgM isotype refers to an antibody having a KD of 10⁻⁷ M or less, or 10⁻⁸ M or less. In one aspect, the anti-OprF/I antibodies or antigen binding fragments thereof described herein have a KD of less than or equal to 1 nM, preferably less than or equal to 200 pM, more preferably less than or equal to 100 pM, and still more preferably less than or equal to 10 pM.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo).

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g. Morrison et al., Proc. Natl. Acad. Sci. USA. 81:6851-6855, 1984; Morrison and Oi. Adv. Immunol., 44:65-92, 1988; Verhoeyen et al., Science, 239:1534-1536.

1988; Padlan, Molec. Immun., 28:489-498. 1991; and Padlan. Molec. Immun., 31:169-217, 1994. Other examples of human engineering technology include, but are not limited to Xoma technology disclosed in U.S. Pat. No. 5,766,886.

The terms “identical” or percent “identity” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., at least 60% identity, optionally at least 65%, 70%, 75%, 80%, 85%, 90%, 95% or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A “comparison window”, as used herein, includes reference to a segment of any one of the number of contiguous positions selected from the group consisting of from 20 to 600. usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman. Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group. 575 Science Or., Madison. WI), or by manual alignment and visual inspection (see. e.g. Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g. Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. The percent identity between two amino acid sequences can also be determined using the algorithm of E. Meyers and W. Miller (Comput. Appl. Biosci. 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453, 1970) algorithm which has been incorporated into the GAP program in the GCG software package (available at www.gcg.com), using either a Blosum62 matrix or a PAM250 matrix, and a gap weight of 16, 14, 12, 10, 8, 6, or 4 and a length weight of 1, 2, 3, 4, 5, or 6. Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated antibody” refers to an antibody that is substantially free of other antibodies having different antigenic specificities (e.g., an isolated antibody that specifically binds an OprF/I agent is substantially free of antibodies that specifically bind antigens other than any of the OprF/I agents). An isolated antibody that specifically binds an OprF/I agent may, however, have cross-reactivity to other antigens. Moreover, an isolated antibody may be substantially free of other cellular material and/or chemicals.

The term “isotype” refers to the antibody class (e.g., IgM, IgE, IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “nucleic acid” is used herein interchangeably with the term “polynucleotide’ and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19.5081. 1991; Ohtsuka et al., J. Biol. Chem. 260:2605-2608, 1985; and Rossolini el. al., Mol. Cell. Probes 8:91-98, 1994).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “treating” includes the administration of compositions such as a vaccine or composition comprising antibodies to prevent or delay the onset of the symptoms, complications, or biochemical indicia of a disease (e.g., cystic fibrosis) or infection (such as e.g. prevent or delay infections in ICU patients or in hospitalized patients or as otherwise herein described), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as an adeno-associated viral vector (AAV, or AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “vaccine” means a biological preparation that improves immunity to a particular infection and/or disease in a particular group of people. A vaccine typically contains an agent that resembles a disease-causing microorganism, and is often made from weakened or killed forms of the microbe or its toxins or is a fragment or fusion of several of such components. The agent stimulates the body's immune system to recognize the agent as foreign, destroy it, and “remember” it, so that the immune system can more easily recognize and destroy any of these microorganisms that it later encounters. Vaccines can be prophylactic (e.g. to prevent or ameliorate the effects of a future infection by any natural or “wild” pathogen), or therapeutic (e.g. vaccines against people with cancer, cystic fibrosis, or people with a transplant that are immunosuppressed may be treated therapeutically).

The term “pharmaceutically effective amount” or “pharmaceutically acceptable amount” of the OprF/I agent of the invention is that amount necessary or sufficient to treat or prevent an infection, disease or state in a patient as described herein, e.g. to reduce the mortality in patient or humans as described herein. The effective amount can vary depending on such factors as the size and weight of the subject, the type of illness, or the particular OprF/I agent of the invention. For example, the choice of the OprF/I agent of the invention can affect what constitutes an “pharmaceutically effective amount”. One of ordinary skill in the art would be able to study the factors contained herein and make the determination regarding the effective amount of the compounds of the invention without undue experimentation.

The term “mcg” is a synonym for microgram.

The term “mortality rate” means in the context of this invention a measure of the number of deaths (in general, or due to a specific cause) in some population, scaled to the size of that population, per unit time. The experimental part of the invention describes e.g. the mortality rate, i.e. the number of death, in the population of ventilated ICU patient in the period of arrival at the ICU (0 day 0) until day 28 at the ICU (also referred to herein as the day 28 mortality).

The term “risk of mortality” or simply “mortality” means in the context of this invention a ratio calculation to standardize the measurement in order to arrive to a comparative measure of mortality in different trials or patient groups. In short, the risk of mortality or mortality is the ratio of the mortality rate in drug treated patients (e.g. in the OprF/I treated patient group) versus the mortality rate in placebo controlled patients times hundred, i.e. risk of mortality (mortality)=mortality rate in drug treated group/mortality rate in placebo group (X100). If mortality about equal to 100=no difference between drug treated and placebo group; if mortality more than 100=mortality rate is higher in the drug treated group than the placebo group; if mortality is less than 100=mortality rate is lower in the drug treated group than in the placebo group.

### The OprF/I Agents of the Invention:

The present invention relates to the use as herein further described of a fusion protein comprising the Pseudomonas aeruginosa outer membrane protein I (full length outer membrane protein I, also called Opr I═SEQ ID NO: 5) which is fused with its amino-terminal end to the carboxy-terminal end of a carboxy-terminal portion of the Pseudomonas aeruginosa outer membrane protein F (full length outer membrane protein F, also called Opr F═SEQ ID NO: 6) such as e.g. SEQ ID NO: 1 and variants thereof (SEQ ID NO: 1 and variants thereof are also referred herein as “OprF/I agent” or “OprF/I agents”).

In a preferred aspect, said fusion protein comprises the carboxy terminal portion of outer membrane protein F with the sequence from amino acid 190 to amino acid 342 of the native OprF protein (SEQ ID NO: 3) or the carboxy terminal portion of outer membrane protein F with the sequence from amino acid 190 to amino acid 350 of the native OprF protein (SEQ ID NO: 2) fused to the amino-terminal end of outer membrane protein I with the sequence from amino acid 21 to amino acid 83 of the native OprI protein (SEQ ID NO: 4).

In a further preferred aspect of the invention, said fusion protein comprises an Ala-(His)₆ tag such as e.g. in the OprF/I agent of SEQ ID NO: 1. Alternatively, the N-terminal tag is selected from Met-, Met-Ala-(His)₆-, Met-Lys-Lys-Thr-Ala-Ile-Ala-Ile-Ala-Val-Ala-Leu-Ala-Gly-Phe-Ala-Thr-Val-Ala-Gln-Ala-, Met-Lys-Leu-Lys-Asn-Thr-Leu-Gly-Val-Val-ILe-Gly-Ser-Leu-Val-Ala-Ala-Ser-Ala-Met-Asn-Ala-Phe-Ala-, or any other N-terminal sequence disclosed in Table 1 of Gabelsberger et al. (1997) (Gabelsberger, J et al., A Hybrid Outer Membrane Protein Antigen for Vaccination Against Pseudomonas aeruginosa, Behring Inst. Mitt., 1997, 98, 302-314), namely the E. coli OmpT signal peptide or the E. chrysanthemii PeIB signal peptide. It is also possible that a spacer, preferably a Ser-Thr-Gly-Ser-spacer, between the tag and the N-terminus of the OprF/I fusion protein is located. A particularly preferred OprF/I fusion protein contains an Ala-(His)₆-N-terminus because the fusion protein can easily be purified by immobilized metal affinity chelate chromatography as explained below.

A particularly preferred embodiment of the present invention is a mixture, in particular a complex, of OprF/I fusion proteins, each of the OprF/I fusion proteins comprises a portion of the Pseudomonas aeruginosa outer membrane protein F which is fused with its carboxy terminal end to a portion of the amino terminal end of the Pseudomonas aeruginosa out membrane protein I, wherein said portion of the Pseudomonas aeruginosa outer membrane protein F comprises the amino acids 190-342 of native Pseudomonas aeruginosa outer membrane protein F and wherein said portion of the Pseudomonas aeruginosa outer membrane protein I comprises the amino acids 21-83 of native of the Pseudomonas aeruginosa outer membrane protein I, and each of the OprF/I fusion proteins contains an Ala-(His)₆-N-terminus, said mixture containing, in particular in the form of a trimer,

(a) an OprF/I fusion protein having only a Cys18-Cys27-bond (SEQ ID NO: 11),
(b) an OprF/I fusion protein having a Cys18-Cys27-bond and a Cys33-Cys47-bond (SEQ ID NO: 12), and/or
(c) an OprF/I fusion protein having a Cys18-Cys47-bond and a Cys27-Cys33-bond (SEQ ID NO: 13).

The amino acid numbering is according to the amino acid sequence of SEQ ID NO: 1. The purity of said mixture is at least about 75%, preferably at least about 80% to about 90%, in particular at least about 85%, e.g. 75% to 90% or 85% to 90% compared to the whole protein content of the mixture as preferably measured by RP-HPLC.

A particular advantage of the present invention is that the OprF/I fusion protein does not form undesired aggregates, in particular high molecular weight aggregates, but preferably trimers. Interestingly, the OprF/I fusion protein trimers have a rather elongated shape instead of a globular shape, and a high hydrodynamic radius, in particular with a calculated Stokes-radius of 5.6 nm. The trimer was stable in solution e.g. under physiological conditions such as e.g. pH around 7 and room temperature, i.e. no dissociation was monitored.

Therefore, another aspect of the present invention is a trimeric OprF/I fusion protein comprising a portion of the Pseudomonas aeruginosa outer membrane protein F which is fused with its carboxy terminal end to a portion of the amino terminal end of the Pseudomonas aeruginosa outer membrane protein I, wherein said portion of the Pseudomonas aeruginosa outer membrane protein F comprises the amino acids 190-342 of native Pseudomonas aeruginosa outer membrane protein F and wherein said portion of the Pseudomonas aeruginosa outer membrane protein I comprises the amino acids 21-83 of native of the Pseudomonas aeruginosa outer membrane protein I, or an immunogenic variant thereof having at least 85%, preferably 90%, in particular 95% identity to the amino acid sequence of SEQ ID NO: 1.

Preferably the trimeric OprF/I fusion protein possesses the same disulfide bonds as explained above. In addition, the trimeric OprF/I fusion protein(s) can be present in a mixture as also explained above.

In a further aspect the OprF/I agent may consist of or comprise a variant of any of the sequences specified herein, such as SEQ ID NOs: 1-13, especially SEQ ID NO: 1. In an embodiment is a functional active variant and/or has at least 50% sequence identity to the sequences specified above, especially at least 60%, more especially at least 70%, preferably at least 80%, more preferably at least 85%, still more preferably at least 90%, even more preferably at least 95%, most preferably 99% sequence identity. A variant is regarded as a functional active variant if it exhibits the biological activity of the sequence from which it is derived, particularly, if the activity of the variant amounts to at least 10%, preferably at least 25%, more preferably at least 50%, even more preferably at least 70%, still more preferably at least 80%, especially at least 90%, particularly at least 95%, most preferably at least 99% of the activity of the sequence without sequence alterations. Activity could be tested as described in the “Experimental Part” or in corresponding animal studies.

In a further preferred aspect, the present invention encompasses an immunogenic variant of the sequences specified herein, such as SEQ ID NO: 1-13, especially SEQ ID NO: 1, which has at least 50% sequence identity, especially at least 60%, more especially at least 70%, preferably at least 80%, more preferably at least 85%, still more preferably at least 90%, even more preferably at least 95%, most preferably 99% sequence identity to the amino acid sequence of SEQ ID NO: 1 with the proviso that the specified cysteine residues forming the disulfide bonds specified above are maintained; and which shows in vivo immunogenicity, e.g. in the BALB/c mouse model, e.g. have an ED50 value of 10 μg of lower, more preferably an ED50 value of 5 μg or lower such as e.g. 4 μg or lower, 3 μg or lower or 2 μg or lower (see example section).

Accordingly, the OprF/I agent may be characterized as a fusion protein comprising or consisting of the Pseudomonas aeruginosa outer membrane protein I fused with its amino-terminal end to the carboxy-terminal end of a carboxy-terminal portion of the Pseudomonas aeruginosa outer membrane protein F, particularly
(i) wherein the Pseudomonas aeruginosa outer membrane protein I is the full length outer membrane protein I; especially SEQ ID NO: 5;
(ii) wherein the Pseudomonas aeruginosa outer membrane protein F is the full length outer membrane protein F, especially SEQ ID NO: 6;
(iii) wherein the OprF/I agent comprises or consist of i) SEQ ID NO: 2, 3, 7 to 10, and/or ii) SEQ ID NO: 4;
(iv) wherein the OprF/I agent consists of or comprises SEQ ID NO: 1;
(v) wherein the OprF/I agent is a functional active variant and/or has at least 50% sequence identity to SEQ ID NO: 1, especially at least 60%, more especially at least 70%, preferably at least 80%, more preferably at leasr 85%, still more preferably at least 90%, even more preferably at least 95%, most preferably 99% sequence identity;
(vi) wherein the OprF/I agent comprises the carboxy terminal portion of outer membrane protein F with the sequence from amino acid 190 to amino acid 342 of the native OprF protein (SEQ ID NO: 3) or the carboxy terminal portion of outer membrane protein F with the sequence from amino acid 190 to amino acid 350 of the native OprF protein (SEQ ID NO: 2);
(vii) wherein the OprF/I agent comprises the amino-terminal end of outer membrane protein I with the sequence from amino acid 21 to amino acid 83 of the native OprI protein (SEQ ID NO: 4);
(viii) wherein the OprF/I agent comprises or consists of the carboxy terminal portion of outer membrane protein F with the sequence from amino acid 190 to amino acid 342 of the native OprF protein (SEQ ID NO: 3) or the carboxy terminal portion of outer membrane protein F with the sequence from amino acid 190 to amino acid 350 of the native OprF protein (SEQ ID NO: 2) fused to the amino-terminal end of outer membrane protein I with the sequence from amino acid 21 to amino acid 83 of the native OprI protein (SEQ ID NO: 4);
(ix) wherein the OprF/I agent forms a trimer comprising or consisting of a polypeptide with SEQ ID NO: 1 or functionally active variants thereof with at least 85% identity to SEQ ID NO: 1;
(x) wherein the OprF/I agent forms a trimer comprising or consisting of a polypeptide with SEQ ID NO: 1 or functionally active variants thereof with at least 85% identity to SEQ ID NO: 1 and wherein said polypeptide or variant thereof have either a) a Cys18-Cys27-bond (see e.g. SEQ ID NO: 11), b) a Cys18-Cys27-bond and a Cys33-Cys47-bond (see e.g. SEQ ID NO: 12), or c) Cys18-Cys47-bond and Cys27-Cys33-bond (see e.g. SEQ ID NO: 13); and/or
(xi) wherein the fusion protein comprises a Ala-(His)₆ tag such as e.g. in the OprF/I agent of SEQ ID NO: 1, 11 to 13.

Another aspect of the present inventions concerns an antibody or antibody derivative which specifically binds the above-specified OprF/I fusion protein(s) and/or the trimeric forms thereof. The antibody is either polyclonal or monoclonal, preferably it is a monoclonal antibody. The term “antibody derivative” is understood as also meaning antigen-binding portions of the inventive antibody, prepared by genetic engineering and optionally modified antibodies, such as, for example, chimeric antibodies, humanized antibodies, multifunctional antibodies, bi- or oligospecific antibodies, single-stranded antibodies, F(ab) or F(ab)₂ fragments, which are all well known for a person skilled in the art.

The invention includes isolated antibodies and antigen-binding portions thereof that selectively bind trimers of OprF/I fusion proteins as described herein. As used herein with respect to the binding of trimers of OprF/I fusion proteins by the antibodies and antigen-binding portions, “selectively binds” means that an antibody (binding fragment thereof) preferentially binds to a trimer of OprF/I fusion proteins (e.g., with greater avidity and/or binding affinity) than to an OprF/I fusion protein monomer. In preferred embodiments, the antibodies of the invention and antigen-binding portions thereof bind to a trimer of OprF/I fusion proteins with an avidity and/or binding affinity that is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 500-fold, 1000-fold or more than that exhibited by the antibody and binding fragments thereof for an OprF/I fusion protein monomer. Preferably, the antibody selectively binds trimers of OprF/I fusion proteins, and not OprF/I fusion protein monomers, i.e., substantially exclusively binds to trimers of OprF/I fusion proteins, or specifically binds trimers of OprF/I fusion proteins without substantial binding to OprF/I fusion protein monomers.

In some embodiments, the isolated antibodies or antigen-binding portions thereof bind to a trimer-specific epitope. Generally, antibodies or antigen-binding portions thereof that bind to a trimer-specific epitope preferentially bind a trimer of OprF/I fusion proteins rather than a OprF/I fusion protein monomer. To determine if a selected antibody binds preferentially (i.e., selectively and/or specifically) to a trimer of OprF/I fusion proteins, each antibody can be tested in comparative assays (e.g., a surface plasmon resonance (SPR) assay such as BiaCore or immunoprecipitation followed by Western blotting) using trimers of OprF/I fusion proteins and OprF/I fusion protein monomers. A comparison of the results will indicate whether the antibodies bind preferentially to the trimer or to the monomer.

Accordingly, the OprF/I agent may be characterized as an antibody or antigen-binding portion thereof directed against the hybrid protein comprising the Pseudomonas aeruginosa outer membrane protein I fused with its amino-terminal end to the carboxy-terminal end of a carboxy-terminal portion of the Pseudomonas aeruginosa outer membrane protein F, particularly
(i) wherein the Pseudomonas aeruginosa outer membrane protein I is the full length outer membrane protein I; especially SEQ ID NO: 5;
(ii) wherein the Pseudomonas aeruginosa outer membrane protein F is the full length outer membrane protein F, especially SEQ ID NO: 6;
(iii) wherein the OprF/I agent consists of or comprises SEQ ID NO: 1;
(iv) wherein the OprF/I agent is a functional active variant and/or has at least 85% sequence identity to SEQ ID NO: 1;
(v) wherein the OprF/I agent forms a trimer comprising or consisting of a polypeptide with SEQ ID NO: 1 or functional active variants thereof with at least 85% identity to SEQ ID NO: 1;
(vi) wherein the OprF/I agent forms a trimer comprising or consisting of a polypeptide with SEQ ID NO: 1 or functionally active variants thereof with at least 85% identity to SEQ ID NO: 1 and wherein said polypeptide or variant thereof have either a) a Cys18-Cys27-bond (see e.g. SEQ ID NO: 11), b) a Cys18-Cys27-bond and a Cys33-Cys47-bond (see e.g. SEQ ID NO: 12), or c) Cys18-Cys47-bond and Cys27-Cys33-bond (see e.g. SEQ ID NO: 13);
(vii) wherein the OprF/I agent comprises or consist of i) SEQ ID NO: 2, 3, 7 to 10, and/or ii) SEQ ID NO: 4;
(viii) wherein the OprF/I agent comprises the carboxy terminal portion of outer membrane protein F with the sequence from amino acid 190 to amino acid 342 of the native OprF protein (SEQ ID NO: 3) or the carboxy terminal portion of outer membrane protein F with the sequence from amino acid 190 to amino acid 350 of the native OprF protein (SEQ ID NO: 2);
(ix) wherein the OprF/I agent comprises the amino-terminal end of outer membrane protein I with the sequence from amino acid 21 to amino acid 83 of the native OprI protein (SEQ ID NO: 4);
(x) wherein the OprF/I agent comprises or consists of the carboxy terminal portion of outer membrane protein F with the sequence from amino acid 190 to amino acid 342 of the native OprF protein (SEQ ID NO: 3) or the carboxy terminal portion of outer membrane protein F with the sequence from amino acid 190 to amino acid 350 of the native OprF protein (SEQ ID NO: 2) fused to the amino-terminal end of outer membrane protein I with the sequence from amino acid 21 to amino acid 83 of the native OprI protein (SEQ ID NO: 4).

The present invention further relates to the use as herein further described of a fusion protein comprising the Pseudomonas aeruginosa outer membrane protein I which is fused with its amino terminal end to the carboxy-terminal end of a carboxy-terminal portion of the Pseudomonas aeruginosa outer membrane protein OprF, wherein said carboxy-terminal portion comprises one or more of the surface-exposed B-cell epitopes SEE 1, SEE 2, SEE 3 and SEE 4. These B-cell epitopes are located at the following amino acid (aa) positions of the OprF: SEE 1=aa 212-240 (SEQ ID NO: 7), SEE 2=aa 243-256 (SEQ ID NO: 8), SEE 3=aa 285-298 (SEQ ID NO: 9) and SEE 4=aa 332-350 (SEQ ID NO: 10) (see Hughes et al. (1992), Infect. Immun. 60, pp. 3497-3503).

Another aspect of the present invention is the use as herein further described of a pharmaceutical composition comprising at least one of the above-mentioned fusion proteins.

Another aspect of the present invention is the use as herein further described of a vaccine comprising at least one of the above-mentioned fusion proteins.

Moreover, the present invention relates to the use as herein further described of a monoclonal or polyclonal antibody (including an antibody derivative described above) directed to one or more of the above fusion proteins. These antibodies may also be used in a pharmaceutical composition in order to confer passive protection against an infection by e.g. Pseudomonas aeruginosa to a subject and thus be useful and indicated for use also in an acute setting as herein further described.

Said OprF/I agents and method of making them are also described e.g. for the antigens in EP717106, Von Specht et al. Infection and Immunity, May 1995, p. 1855-1862) and in the experimental part herein. In short, said OprF/I agents may be produced according to a process, which comprises bringing about the expression of a nucleic acid that is coding for said fusion protein in pro- or eukaryotic cells. An OprF/I agent such as e.g. SEQ ID NO: 1 may be formulated e.g. as an injectable (such as for intramuscular or intravenous, preferably intramuscular administration) in a dose of 100 mcg in a physiological salt solution (0.81% weight per volume) with or without aluminium hydroxide (400 mcg). The making of the antibodies once a specific antigen is known is well known in the art and e.g. in the case of the production and making of a fully human antibody may be done in accordance of the method as described in WO2008055795 and WO04102198.

### The Novel and Inventive Uses for the OprF/I Agents of the Invention:

In accordance with the particular findings of the present invention, the following novel and inventive methods and/or uses are provided:
1.1 A method of reducing mortality in a ventilated intensive care unit patient such as mechanically ventilated intensive care unit patient comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.2 A method of reducing mortality in a cystic fibrosis patient comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.3 A method of reducing mortality in a burn victim such as a first, second or third degree burn victim, preferably in a third degree burn victim, comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.4 A method of reducing mortality in cancer or transplant patients who are immunosuppressed comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.5 A method of reducing mortality in a intensive care unit patient comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.6 A method of reducing mortality in a hospitalized patient comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.7 A method of reducing mortality in a patient admitted to the intensive care unit with the need for mechanical ventilation for more than 48 hours comprising administering to said patient an effective amount (such as a pharmaceutically effective amount) of a pharmaceutical composition comprising an OprF/I agent, e.g. SEQ ID NO: 1 and optionally a pharmaceutically acceptable excipient;
1.8 A method of reducing mortality in a human or non-human animal who will be operated or who is planning to be operated comprising administering to said human or non-human animal an effective amount (such as a pharmaceutically effective amount) of a pharmaceutical composition comprising an OprF/I agent, e.g. SEQ ID NO: 1 and optionally a pharmaceutically acceptable excipient, preferably at least 2 weeks before the planned operation;
1.9 A method of reducing mortality in a human that is at risk to be admitted to the intensive care unit such as a human who is doing extreme sports (such as base jumping, bungee jumping, gliding, hang gliding, high wire, ski jumping, sky diving, sky surfing, sky flying, indoor climbing, adventure racing, aggressive inline skating, BMX, caving, extreme motocross, extreme skiing, freestyle skiing, land and ice yachting, mountain biking, mountain boarding, outdoor climbing, sandboarding, skateboarding, snowboarding, snowmobiling, speed biking, speed skiing, scootering, barefoot waterskiing, cliff diving, free-diving, jet skiing, open water swimming, powerboat racing, round the world yacht racing, scuba diving, snorkeling, speedsailing, surfing, wakeboarding, whitewater kayaking, windsurfing) comprising administering to said human an effective amount (such as a pharmaceutically effective amount) of a pharmaceutical composition comprising an OprF/I agent, e.g. SEQ ID NO: 1 and optionally a pharmaceutically acceptable excipient, preferably at least 2 weeks before the planned extreme sport event;
1.10 A method of reducing mortality in a human, in particular a human of any age, e.g. of age 2 or older, comprising administering to said human an effective amount (such as a pharmaceutically effective amount) of a pharmaceutical composition comprising an OprF/I agent, e.g. SEQ ID NO: 1 and optionally a pharmaceutically acceptable excipient, preferably wherein said method additionally comprises regular booster vaccinations;
1.11 A method of reducing mortality in a human with any kind of infection, comprising administering to said human an effective amount (such as a pharmaceutically effective amount) of a pharmaceutical composition comprising an OprF/I agent, e.g. SEQ ID NO: 1 and optionally a pharmaceutically acceptable excipient, preferably wherein said method additionally comprises regular booster vaccinations;
1.12 A method as defined above, wherein the OprF/I agent is selected from the group consisting of a polypeptide consisting of i) SEQ ID NO: 2, 3, 7 to 10, and ii) SEQ ID NO: 4; SEQ ID NO: 1 SEQ ID NO: 11 to 13; and an antibody or fragment thereof directed against said polypeptide or SEQ ID NO: 1;
1.13 A method as defined above, wherein the pharmaceutical composition is a vaccine;
1.14 A method as defined above, wherein the pharmaceutical composition is a vaccine that is non-adjuvanted.
1.15 A method as defined above, comprising co-administration of a first drug substance, said first drug substance being an effective amount (such as a pharmaceutically effective amount) of a vaccine comprising an OprF/I agent, e.g. SEQ ID NO: 1, and a second drug substance, said second drug substance being an effective amount (such as a pharmaceutically effective amount) of an agent selected from the group consisting of antibiotics such as intravenous antibiotics and other drug substances improving the state of the patient, human, or non-human animal in particular in regards to reducing the risk of mortality;
1.16 A method as defined above, wherein the mortality is lower than 100.
1.17 A method as defined above, wherein the mortality e.g. in an ICU patient, preferably in ventilated ICU patients, is lower than 95, preferably 90, more preferably 85, more preferably 80, more preferably 75, more preferably 70, more preferably 65, even more preferably lower than 60, most preferably lower than 55.
1.18 A method as defined above, wherein the OprF/I agent is a protein complex comprising (or consisting at least 80%, preferably 85%, more preferably 90% of) three OprF/I agents with SEQ ID NO: 1 or an immunogenic variant thereof having at least 85%, preferably 90%, in particular 95% identity to the amino acid sequence of SEQ ID NO:1;
1.19 A method as defined above, wherein the protein complex comprises OprF/I agents that are selected from the group consisting of
   (a) the OprF/I agent of SEQ ID NO: 1 with a Cys18-Cys27-bond (SEQ ID NO: 11), and
   (b) the OprF/I agent of SEQ ID NO: 1 with a Cys18-Cys27-bond and Cys33-Cys47-bond (SEQ ID NO: 12), and
   (c) the OprF/I agent of SEQ ID NO: 1 with a Cys18-Cys47-bond and Cys27-Cys33-bond (SEQ ID NO: 13),
   or an immunogenic variant thereof having at least 85%, preferably 90%, in particular 95% identity to the amino acid sequence of SEQ ID NO: 1, and the same disulphide bond pattern as specified in (a), (b) or (c);
   preferably the sum of a) the OprF/I agent of SEQ ID NO: 1 with a Cys18-Cys27-bond (SEQ ID NO: 11), b) the OprF/I agent of SEQ ID NO: 1 with a Cys18-Cys27-bond and Cys33-Cys47-bond (SEQ ID NO: 12), and c) the OprF/I agent of SEQ ID NO: 1 with a Cys18-Cys47-bond and Cys27-Cys33-bond (SEQ ID NO: 13) is equal or greater than 75%.

Suitable second drug substances may include e.g. antibiotics such as i) an antimicrobial compound, e.g. penicillins, cephalosporins, polymixins, quinolones, sulfonamides, aminoglycosides, macrolides, tetracyclines, daptomycins, tigecyclines, linezolids; ii) an antifungal compound, e.g. polyene antimycotics, natamycin, rimocidin, filipin, nyastatin, amphotericin B, candicin, hamycin; iii) an antibody directed against OprF/I as defined herein in case the OprF/I agent of the first substance is an antigen.

The terms “co-administration” or “combined administration” or the like as utilized herein are meant to encompass administration of the selected therapeutic or prophylactic agents to a single patient, and are intended to include treatment or prophylactic regimens in which the agents are not necessarily administered by the same route of administration or at the same time.

As alternative to the above the present invention also provides:
2. An OprF/I agent, e.g. SEQ ID NO: 1, for use in any method as defined under 1.1 to 1.19 above; or
3. An OprF/I agent, e.g. SEQ ID NO: 1, for use in the preparation of a pharmaceutical composition for use in any method as defined under 1.1 to 1.19 above; or
4. A pharmaceutical composition for use in any method as defined under 1.1 to 1.19 above comprising an OprF/I agent, e.g. SEQ ID NO: 1, together with one or more pharmaceutically acceptable diluents or carriers.
5.1 A pharmaceutical combination comprising:
   a) a first agent which is a vaccine comprising an OprF/I agent, e.g. SEQ ID NO: 1, wherein said vaccine is optionally non-adjuvanted, and
   b) a co-agent which is an antimicrobial or antifungal compound, e.g. as disclosed above.
5.2 A pharmaceutical combination comprising:
   a) a first agent which is antibody or fragment thereof directed against an OprF/I agent, e.g. SEQ ID NO: 1, and
   b) a co-agent which is an antimicrobial or antifungal compound, e.g. as disclosed above.

The term “pharmaceutical combination” as used herein means a product that results from the mixing or combining of more than one active ingredient and includes both fixed and non-fixed combinations of the active ingredients. The term “fixed combination” means that the active ingredients, e.g. OprF/I agent and a co-agent, are both administered to a patient simultaneously in the form of a single entity or dosage. The term “non-fixed combination” means that the active ingredients, e.g. OprF/I agent and a co-agent, are both administered to a patient as separate entities either simultaneously, concurrently or sequentially with no specific time limits, wherein such administration provides therapeutically effective levels of the 2 compounds in the body of the patient.

Utility of the OprF/I agents, e.g. SEQ ID NO: 1, alone or in combinations with other drug substances as described herein or elsewhere, in the reduction of the mortality rate in suitable groups such as hospitalized patients, Cystic fibrosis patients, ICU patients, ICU patients that are mechanically ventilated, burn victims such as severe burn patients, cancer and/or transplant patients who are immunosuppressed or will be immunosuppressed, humans or non-human animals that will be or are planning to be operated, e.g. as hereinabove specified, may be further demonstrated in animal test methods as well as in further clinical trials, for example in accordance with the methods hereinafter described in the experimental part.

Pharmaceutical composition of the invention including vaccines can be prepared in accordance with methods well known and routinely practiced in the art (see e.g. Remington: The Science and Practice of Pharmacy, Mack Publishing Co. 20^{th} ed. 2000; and Ingredients of Vaccines-Fact Sheet from the Centers for Disease Control and Prevention, e.g. adjuvants and enhancers such as alum to help the vaccine improve its work, preservatives and stabilizers to help the vaccine remain unchanged (e.g. albumin, phenols, glycine)). Pharmaceutical compositions are preferably manufactured under GMP conditions. Typically a pharmaceutically effective dose of the OprF/I agent is employed in the pharmaceutical composition of the invention. The OprF/I agents are formulated into pharmaceutically acceptable dosage forms by conventional methods known to those of skill in the art. Dosage regimens are adjusted to provide the optimum desired response (e.g. the therapeutic or prophylactic response). For example, two unit dosage forms may be e.g. administered in case of a vaccine (such as the vaccine in the experimental part, i.e. a vaccine comprising SEQ ID NO: 1), or several divided doses (e.g. in the case of an antibody composition) may be administered over time or the dose may be proportionally reduced or increased as indicated by the exigencies of the therapeutic situation. It is especially advantageous to formulate parenteral compositions in dosage unit forms for ease of administration and uniformity of dosage. Dosage unit forms as used herein (e.g. 100 mcg unit form of SEQ ID NO: 1) refers to physically discrete units suited as unitary dosages for the subjects to be treated; each unit contains a predetermined quantity of active OprF/I agent calculated to produce the desired pharmaceutical effect in association with the required pharmaceutical carrier or excipient.

Actual dosage levels of the active ingredients in the pharmaceutical compositions of the present invention can be varied so as to obtain an amount of the active ingredient which is effective to achieve the desired pharmaceutical response for a particular patient, composition, and mode of administration, without being toxic to the patient. The selected dosage level depends upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the OprF/I agents of the invention employed in the pharmaceutical composition at levels lower than that required to achieve the desired therapeutic effect and gradually increase the dosage until the desired effect is achieved. In general, effective doses of the compositions of the present invention, for the prophylactic and therapeutic treatment of groups of people as described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy. For systemic administration with an OprF/I agent such as a vaccine or an antibody of the invention, the dosage ranges from about 0.01 to 100 mcg/kg, and more usually 1 to 15 mcg/kg, of the host body weight. An exemplary treatment regime entails systemic administration e.g. twice or once for a vaccine or once per every two weeks or once a month or once every 3 to 6 months for an antibody treatment. An exemplary treatment regime entails systemic administration twice at day 0 and day 7 for the vaccine consisting of 100 mcg SEQ ID NO: 1 and 0.81% weight per volume NaCl in water.

In a further aspect of the invention, the invention provides the following pharmaceutical compositions:
1.1. A pharmaceutical composition for use in reducing mortality in a ventilated intensive care unit patient such as mechanically ventilated intensive care unit patient comprising an OprF/I agent as described herein, e.g. SEQ ID NO: 1 and optionally a pharmaceutically acceptable excipient;
1.2 A pharmaceutical composition for use in reducing mortality in a cystic fibrosis patient comprising an OprF/I agent as described herein, e.g. SEQ ID NO: 1 and optionally a pharmaceutically acceptable excipient;
1.3 A pharmaceutical composition for use in reducing mortality in a burn victim such as a first, second or third degree burn victim, preferably in a third degree burn victim, comprising an OprF/I agent as described herein, e.g. SEQ ID NO: 1 and optionally a pharmaceutically acceptable excipient;
1.4 A pharmaceutical composition for use in reducing mortality in cancer or transplant patients who are immunosuppressed comprising an OprF/I agent, e.g. SEQ ID NO: 1 and optionally a pharmaceutically acceptable excipient;
1.5 A pharmaceutical composition for use in reducing mortality in a intensive care unit patient comprising an OprF/I agent as described herein, e.g. SEQ ID NO: 1 and optionally a pharmaceutically acceptable excipient;
1.6 A pharmaceutical composition for use in reducing mortality in a hospitalized patient comprising an OprF/I agent as described herein, e.g. SEQ ID NO: 1 and optionally a pharmaceutically acceptable excipient;
1.7 A pharmaceutical composition for use in reducing mortality in a patient admitted to the intensive care unit with the need for mechanical ventilation for more than 48 hours comprising an OprF/I agent as described herein, e.g. SEQ ID NO: 1 and optionally a pharmaceutically acceptable excipient;
1.8 A pharmaceutical composition for use in reducing mortality in a human or non-human animal who will be operated or who is planning to be operated comprising an OprF/I agent as described herein, e.g. SEQ ID NO: 1 and optionally a pharmaceutically acceptable excipient, preferably at least 2 weeks before the planned operation;
1.9 A pharmaceutical composition for use in reducing mortality in a human that is at risk to be admitted to the intensive care unit such as a human who is doing extreme sports (such as base jumping, bungee jumping, gliding, hang gliding, high wire, ski jumping, sky diving, sky surfing, sky flying, indoor climbing, adventure racing, aggressive inline skating, BMX, caving, extreme motocross, extreme skiing, freestyle skiing, land and ice yachting, mountain biking, mountain boarding, outdoor climbing, sandboarding, skateboarding, snowboarding, snowmobiling, speed biking, speed skiing, scootering, barefoot waterskiing, cliff diving, free-diving, jet skiing, open water swimming, powerboat racing, round the world yacht racing, scuba diving, snorkeling, speedsailing, surfing, wakeboarding, whitewater kayaking, windsurfing) comprising an OprF/I agent as described herein, e.g. SEQ ID NO: 1 and optionally a pharmaceutically acceptable excipient, preferably administered at least 2 weeks before the planned extreme sport event;
1.10 A pharmaceutical composition for use in reducing mortality in a human, in particular a human of any age, e.g. of age 2 or older, comprising an OprF/I agent as described herein, e.g. SEQ ID NO: 1 and optionally a pharmaceutically acceptable excipient;
1.11 A pharmaceutical composition for use in reducing mortality in a human with any kind of infection, comprising an OprF/I agent as described herein, e.g. SEQ ID NO: 1 and optionally a pharmaceutically acceptable excipient;
1.12 A pharmaceutical composition for use as defined above, wherein the OprF/I agent is selected from the group consisting of a polypeptide consisting of i) SEQ ID NO: 2, 3, 7 to 10, and ii) SEQ ID NO: 4; SEQ ID NO: 1; SEQ ID NO: 11 to 13; and an antibody or fragment thereof directed against said polypeptide or SEQ ID NO: 1;
1.13 A pharmaceutical composition for use as defined above, wherein the pharmaceutical composition is a vaccine;
1.14 A pharmaceutical composition for use as defined above, wherein the pharmaceutical composition is a vaccine that is non-adjuvanted or at least not adjuvanted by alum.
1.15 A pharmaceutical composition for use as defined above, comprising co-administration of a first drug substance, said first drug substance being an effective amount (such as a pharmaceutically effective amount) of a vaccine comprising an OprF/I agent as described herein, e.g. SEQ ID NO: 1, and a second drug substance, said second drug substance being an effective amount (such as a pharmaceutically effective amount) of an agent selected from the group consisting of antibiotics such as intravenous antibiotics and other drug substances improving the state of the patient, human, or non-human animal in particular in regards to reducing the risk of mortality;
1.16 A pharmaceutical composition for use as defined above, wherein the mortality is lower than 100.
1.17 A pharmaceutical composition for use as defined above, wherein the mortality e.g. in an ICU patient, preferably in ventilated ICU patients, is lower than 95, preferably 90, more preferably 85, more preferably 80, more preferably 75, more preferably 70, more preferably 65, even more preferably lower than 60, most preferably lower than 55.

The antibody composition is usually administered on multiple occasions. Intervals between single dosages can be weekly, monthly or yearly. Intervals can also be irregular as indicated by measuring blood levels of antibody in the patient. In some methods of systemic administration, dosage is adjusted to achieve a plasma antibody concentration of 1-1000 μg/ml and in some methods 25-500 μg/ml. Alternatively, antibody can be administered as a sustained release formulation, in which case less frequent administration is required. Dosage and frequency vary depending on the half-life of the antibody in the patient. In general, humanized antibodies show longer half life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

TABLE 1
Sequences

| SEQ | | |
|---|---|---|
| ID | Alternative | |
| NO: | name(s) | Amino acid sequence |
| 1 | Ala-(His)6- | AHHHHHHAPAPEPVADVCSDSDNDGVCDNVDKCPDTPANVTVDANG |
| | OprF190-342- | CPAVAEVVRVQLDVKFDFDKSKVKENSYADIKNLADFMKQYPSTSTTV |
| | OprI21-83 | EGHTDSVGTDAYNQKLSERRANAVRDVLVNEYGVEGGRVNAVGYGE |
| | | SRPVADNATAEGRAINRRVESSHSKETEARLTATEDAAARAQARADEA |
| | | YRKADEALGAAQKAQQTADEANERALRMLEKASRK |
| 2 | OprF190-350 | APAPEPVADVCSDSDNDGVCDNVDKCPDTPANVTVDANGCPAVAEVV |
| | | RVQLDVKFDFDKSKVKENSYADIKNLADFMKQYPSTSTTVEGHTDSVG |
| | | TDAYNQKLSERRANAVRDVLVNEYGVEGGRVNAVGYGESRPVADNAT |
| | | AEGRAINRRVEAEVEAEAK |
| 3 | OprF190-342 | APAPEPVADVCSDSDNDGVCDNVDKCPDTPANVTVDANGCPAVAEVV |
| | | RVQLDVKFDFDKSKVKENSYADIKNLADFMKQYPSTSTTVEGHTDSVG |
| | | TDAYNQKLSERRANAVRDVLVNEYGVEGGRVNAVGYGESRPVADNAT |
| | | AEGRAINRRVE |
| 4 | OprI21-83 | SSHSKETEARLTATEDAAARAQARADEAYRKADEALGAAQKAQQTAD |
| | | EANERALRMLEKASRK |
| 5 | OprI, NP_251543 | MNNVLKFSALALAAVLATGCSSHSKETEARLTATEDAAARAQARADEA |
| | | YRKADEALGAAQKAQQTADEANERALRMLEKASRK |
| 6 | OprF, | MKLKNTLGVVIGSLVAASAMNAFAQGQNSVEIEAFGKRYFTDSVRNMK |
| | NP_250468 | NADLYGGSIGYFLTDDVELALSYGEYHDVRGTYETGNKKVHGNLTSLD |
| | | AIYHFGTPGVGLRPYVSAGLAHQNITNINSDSQGRQQMTMANIGAGLK |
| | | YYFTENFFAKASLDGQYGLEKRDNGHQGEWMAGLGVGFNFGGSKAA |
| | | PAPEPVADVCSDSDNDGVCDNVDKCPDTPANVTVDANGCPAVAEVVR |
| | | VQLDVKFDFDKSKVKENSYADIKNLADFMKQYPSTSTTVEGHTDSVGT |
| | | DAYNQKLSERRANAVRDVLVNEYGVEGGRVNAVGYGESRPVADNATA |
| | | EGRAINRRVEAEVEAEAK |
| 7 | OprF212-240 | NVDKCPDTPANVTVDANGCPAVAEVVRVQL |
| 8 | OprF243-256 | KFDFDKSKVKENSY |
| 9 | OprF285-298 | TDAYNQKLSERRAN |
| 10 | OprF332-350 | EGRAINRRVEAEVEAEAK |
| 11 | Ala-(His)6- | AHHHHHHAPAPEPVADVCSDSDNDGVCDNVDKCPDTPANVTVDANG |
| | OprF190-342- | CPAVAEVVRVQLDVKFDFDKSKVKENSYADIKNLADFMKQYPSTSTTV |
| | OprI21-83, | EGHTDSVGTDAYNQKLSERRANAVRDVLVNEYGVEGGRVNAVGYGE |
| | disulfide bond | SRPVADNATAEGRAINRRVESSHSKETEARLTATEDAAARAQARADEA |
| | between residues | YRKADEALGAAQKAQQTADEANERALRMLEKASRK |
| | 18-27 (both bold) | |
| 12 | Ala-(His)6- | AHHHHHHAPAPEPVADVCSDSDNDGVCDNVDKCPDTPANVTVDANG |
| | OprF190-342- | CPAVAEVVRVQLDVKFDFDKSKVKENSYADIKNLADFMKQYPSTSTTV |
| | OprI21-83, | EGHTDSVGTDAYNQKLSERRANAVRDVLVNEYGVEGGRVNAVGYGE |
| | disulfide bonds | SRPVADNATAEGRAINRRVESSHSKETEARLTATEDAAARAQARADEA |
| | between residues | YRKADEALGAAQKAQQTADEANERALRMLEKASRK |
| | 18-27 (both bold) | |
| | and 33-47 (both | |
| | italic) | |
| 13 | Ala-(His)6- | AHHHHHHAPAPEPVADVCSDSDNDGVCDNVDKCPDTPANVTVDANG |
| | OprF190-342- | CPAVAEVVRVQLDVKFDFDKSKVKENSYADIKNLADFMKQYPSTSTTV |
| | OprI21-83, | EGHTDSVGTDAYNQKLSERRANAVRDVLVNEYGVEGGRVNAVGYGE |
| | disulfide bonds | SRPVADNATAEGRAINRRVESSHSKETEARLTATEDAAARAQARADEA |
| | between residues | YRKADEALGAAQKAQQTADEANERALRMLEKASRK |
| | 18-47 (both bold) | |
| | and 27-33 (both | |
| | italic) | |

### EXAMPLES

Abbreviations

| Abbreviation | Explanation |
|---|---|
| AUC | Analytical ultracentrifugation |
| CV | Column volume |
| DTT | Dithiothreitol |
| DV | Diafiltration volumes |
| DS | Drug substance |
| ED50 | Reverse of the dilution of the samples resulting in 50% |
| | seroconversion rate |
| EGT | Eurogentec |
| gDNA | Genomic DNA |
| GMT | Geometric mean titer |
| GSH | Reduced glutathione |
| GSSG | Oxidized glutathione |
| HCP | Host cell protein |
| HPLC | High performance liquid chromatography |
| ICLL | Intercell |
| IMAC | Immobilized metal affinity chelate chromatography |
| MALDI-ToF | Matrix assisted Lased Desorption Ionization Mass |
| | Spectrometry-Time of Flight |
| MALS | Multi Angle Light Scattering |
| β-ME | Beta-mercaptoethanol |
| PAGE | Polyacrylamide gel Electrophoresis |
| QSHP | Q-Sepharose HP |
| RP | Reversed phase |
| RT | Room temperature (about 20° C.) |
| SCD | Sedimentation Coefficient Distributions |
| SEC | Size exclusion chromatography |
| UF/DF | Ultrafiltration/Diafiltration |

### A. Materials

### General Materials

NaOH (Riedel-de Haen), NaCl (Riedel-de Haen), Tris(hydroxymethyl)aminomethane (Merck KGaA, Darmstadt), L-Cystine (Aldrich), DTT (Sigma), HCl (Merck KGaA), Q-Sepharose® HP (GE Healthcare), DEAE-Sepharose® FF (GE Healthcare). All other materials were of analytical grade if not otherwise stated.

Formulation buffer: Dulbecco's 1×PBS pH 7.4 (H15-002), 1× concentrate (g/L)

| | |
|---|---|
| KCl | 0.2 g/L |
| KH₂PO₄ | 0.2 g/L |
| NaCl | 8.0 g/L |
| Na₂HPO₄ anhydrous | 1.15 g/L |

### Drug Substance

Drug substance used for the clinical trial is the OprF/I agent Ala-(His)₆-OprF 190-342-OprI 21-83 protein (═SEQ ID NO: 1): The fusion protein construct consists of epitopes of two outer membrane proteins of Pseudomonas aeruginosa, OprF and OprI with an N-terminal His 6 tag: Met-Ala-(His)₆-OprF 190-342-OprI 21-83 protein. It is recombinant expressed in E. coli as a 224 AA fusion protein. The N-terminal Met is cleaved off after expression. N-terminal OprF fragment, including the His 6 tag ranges from amino acid A-1 (A) to amino acid 160-E, followed by the OprI fragment at position 161 to 223. Construction of starting materials (i.e. relevant DNA constructs), expression and purification of Ala-(His)₆-OprF 190-342-OprI 21-83 protein and its trimeric conformer comprising the 3 disulfide bond pattern variants is described further below.

### General Methods

### Reduction of Protein Samples

OprF/I fusion protein samples were reduced with β-mercaptoethanol (2.5% v/v final volume, approx. 360 mM final concentration) if not otherwise stated. Samples are incubated at RT for 20 to 30 min to ensure full reduction of disulphide bonds.

### RP-HPLC

For downstream development work an estimation of the specific OprF/I content in IMAC/G50 was necessary to calculate step yields. OprF/I content was determined by RP-HPLC. The HPLC system was calibrated with purified, native (unreduced) OprF/I working standard. The protein content of the working standard was determined by UV 280 nm measurement based on a calculated theoretical extinction coefficient for a 1 mg/mL solution of ε_{0.1%}=0.373. Prior to analysis of IMAC/G50 pools by RP-HPLC, an aliquot was fully reduced by addition of DTT or β-mercaptoethanol (100 mM final concentration) to split up the various aggregated and misfolded (most probably disulfide scrambled) OprF/I variants. The samples were incubated at room temperature for 30 minutes and analyzed by RP-HPLC. After reduction, OprF/I eluted as a single peak compared to the untreated IMAC/G50 pool. The content of reduced OprF/I after IMAC/G50 was calculated by integration of the peak area.

All other samples (e.g. reoxidized OprF/I, fractions from QS-HP etc.) were directly injected without further treatment and the OprF/I concentration was calculated.

Reoxidized samples can be immediately analyzed by RP-HPLC or formation of disulfide bonds can be quenched by acidification to pH 2-3 (˜20 μL 6% HCl per 1 ml reoxidation solution) and stored at 2-8° C. for subsequent analysis.

### Analytical RP-HPLC

Analytical RP-HPLC analysis of samples was performed on a Jupiter C4 column (4.6 mm×150 mm, 300 A, 5 μm, Phenomenex) connected to a Dionex Ultimate 3000 HPLC system. Solvent A was water containing 0.1% TFA, solvent B was acetonitrile containing 0.1% TFA. Separation of peaks was performed by linear gradient elution from 27% B to 37% B in 13 min at a flow rate of 1 mL/min. The column temperature was set to 40° C. Peak detection was performed at 214 nm and 280 nm.

### Preparative RP-HPLC

Preparative RP-HPLC was used for isolation of individual peaks detected by analytical RP-HPLC. Purification was done on a Jupiter C4 column (10 mm×250 mm, 300 A, 5 μm, Phenomenex) connected to an Akta Purifier chromatography system. The stationary phase at preparative scale was the same as the one used at analytical scale. Solvent A was water containing 0.1% TFA, solvent B was 80% acetonitrile in water containing 0.1% TFA. Sample volume was 2 to 4 mL (total protein load <2 mg). Separation of peaks was performed by linear gradient elution from 35% B to 40% B over 8 column volumes at a flow rate of 2.5 mL/min. The column temperature was set to 40° C. Peak detection was done at 280 and 214 nm. Fractions of 0.8 mL were collected and the pH was adjusted to pH˜7 by addition of 0.25 mL 0.1 M sodium phosphate buffer, pH 7.0. Higher quantities (˜0.5 to 2 mg) of P1 to 4 were prepared by several preparative purification runs. After pooling of the desired fractions containing the individual peaks, samples were concentrated approximately 5 times using a 5 kDa ultracentrifugation device (Millipore). Concentrated pools were desalted by PD10 columns (GE Healthcare) and the buffer was exchanged against final drug product formulation buffer (1/10 PBS diluted with 0.9% NaCl, pH ˜7). Final samples containing the isolated OprF/I variants were analyzed for purity and content by RP-HPLC and SEC-HPLC. The relative purity determined by RP-HPLC was at least 90%. Samples were stored at −20° C. until further analysis.

### SDS-PAGE

SDS-PAGE was done on 4-12% NuPAGE gels (Invitrogen) using MES running buffer. Samples were mixed with LDS sampling buffer under reducing or non-reducing conditions and incubated for 5 min at 70° C. if not otherwise stated. Staining was done with colloidal Coomassie or silver stain (Heukeshoven).

### Western Blot Analysis

Western blotting was done with antibodies anti OprF/I 944/5 D5 epitope (1:20000 diluted) and 966/363 E3 epitope (1:10000 diluted).

### pH and Conductivity Measurement

For determination of pH and conductivity of samples and buffers a WTW 720 system was used. Conductivity was measured using the linear temperature compensation mode at 25° C.

### Endotoxin Measurement

Endotoxin measurement was done with a chromogenic LAL-assay (Cambrex). Selected samples were also measured in an external certified laboratory with a conventional gel clot assay (Limulus Amoebocyte Lysate test).

### Host Cell Protein Measurement (HCP)

For quantification of HCPs, a generic E. coli HCP ELISA kit (Cygnus Technologies, Inc.) was used.

### Peptide-Mass Fingerprint and Disulphide Mapping

Purified fractions obtained from preparative RPC were analyzed by LC-MS/MS. Samples were digested with AspN or trypsin without reduction or after reduction and alkylation.

### MALDI-ToF Mass Spectrometry

MALDI-ToF analysis was performed on a Voyager STR 4069 system (Applied Biosystems). Sinapinic acid dissolved in 0.1% TFA/30% AcN was used as sample matrix. DS samples were diluted five-fold with sample matrix and 2 μl were placed on the target. A delayed extraction mode and positive polarity was used. The system was externally calibrated with BSA (Mass calibration kit, Applied Biosystems). For internal calibration Myoglobin (Sigma M-0630, average Mr 16951.5) was spiked into DS samples at a concentration of approximately 100 μg/mL. The mass accuracy for internal calibration can be estimated with approximately ±0.3% (e.g. 24100±72 Da), for external calibration ±0.6% (e.g. 24100±145 Da).

### Native PAGE

The NativePAGE™ Novex® Bis-Tris Gel system is a near neutral pH, pre-cast polyacrylamide mini gel system to perform native (non-denaturing) electrophoresis. Native PAGE of OprF/I fusion protein samples was done on NativePAGE 4-16% Bis-Tris gels (Invitrogen) according to the manufacturers instruction. Sample buffer was 50 mM BisTris, 50 mM NaCl, 16 mM HCl, 10% w/v Glycerol, 0.001% Ponceau S, pH 7.2. Running buffer was 50 mM BisTris, 50 mM Tricine, pH 6.8. Cathode buffer was running buffer including 0.02% Coomassie G-250.

### N-Terminal Sequencing

N-terminal sequencing was carried out using an Applied Biosystems 494HT machine and the method of N-terminal Edman sequencing, where the N-terminal amino acid of the protein was sequentially removed chemically and identified by HPLC. The protein was first immobilized inside the sequencing instrument by either blotting it onto a PVDF membrane or adsorbing it onto a biobrene treated glass fibre filter. Subsequently the bound protein reacted with the Edman reagent, (phenylisothiocyanate, PITC) at high pH. After this reaction, the resulting compound was cleaved off the protein using anhydrous acid. The coupling and cleavage process was repeated for as many times as required. Usually 15 to 20 AA could be analyzed. The cleaved products were converted to their stable phenylthiohydantoins, PTH, with aqueous acid, and then analyzed using the on-board HPLC. Identification of the amino acids was achieved by comparing elution times compared to a standard mixture. Data from the HPLC was collected on a computer for visual calling of the sequence.

### Alkylation of Thiolgroups

Free thiol groups in proteins can be detected by alkylation using iodoacetamide, which reacts selectively with free thiol groups of cysteins to produce carboxamidomethyl cysteine. If free thiol groups are present, these would be covalently blocked resulting in a mass increase of 57 Da per attached iodacetamide molecule.

47 mg iodoacetamide were dissolved in 1 mL 1 M Tris-HCl, pH 8.0 (0.2 M iodoacetamide solution). 200 μL each of purified peak 1, 2 and 3 (protein concentration approximately 200 μg/mL) were mixed with 20 μL of iodoacetamide stock solution (final iodoacetamide concentration ˜0.02M). The OprF/I fusion protein sample (protein concentration approximately 1 mg/mL) was 3 fold diluted with PBS to a final concentration of approximately 330 μg/mL. 30 μL iodacetamide stock solution were added to 300 μL diluted DS. In another experiment the sample was reduced with 5 mM DTT (20 min) before dilution and alkylation. All samples were incubated at room temperature in the dark for 30 min followed by LC-MS analysis.

### Static Light Scattering Analysis

The chromatographic system consisted of an HPLC system from Dionex including an Ultimate 3000 pump and degasser, an Ultimate 3000 autosampler and an Ultimate 3000 column compartment. Column and chromatographic conditions were the same as described for SEC-HPLC. All solvents were filtered through a 0.1 μm Supor Membrane filter (Pall VacuCap 60). An injection volume of 100 μL was used for all samples if not stated otherwise.

Chromatographic detectors included a Dionex Ultimate 3000 photodiode array detector set to 214 nm and 280 nm, a Shodex RI-101 refractive index detector and a DAWN TREOS MALS (multi angle light scattering) detector (Wyatt Technology Corporation), which was used in on-line mode. Chromatographic data collection and analysis was performed using the Chromeleon software package (vers. 6.80, Dionex). Experimental collection and data analysis of the MALS-signals were performed with the ASTRA software package (version 5.3.2.13, Wyatt Technology). Using this software it was possible to collect and subsequently analyze the light scattering signals (3 MALS angles) along with the UV-, and RI-signals.

### Analytical Ultracentrifugation (AUC)

All experiments were performed with a BeckmanCoulter XL-I Analytical Ultracentrifuge at 50.000 rpm and 25° C. Samples were placed in sapphire-capped two-sector titanium centerpieces of 12 mm optical path length. 390 μL of solution and solvent were placed in the sample and reference sectors, respectively. Sedimentation traces were detected by recording local differences in refractive index (interference optics). The samples were analyzed with a ten-fold dilution or without further dilution. Diffusion-corrected Sedimentation Coefficient Distributions (SCD) were calculated using the finite element approach proposed by P. Schuck, NIH (Peter Schuck et al., Biopolymers, Vol 54, Issue 5, pages 328-341, October 2000). The frictional ratio f/f0 was treated as a fitting variable. The density and viscosity of the buffer (phosphate buffered saline, PBS) as well as the partial specific volume (v) of the proteins were calculated from composition with Sednterp. These values were used when calculating the respective SCD.

### Analysis of OprF/I Fusion Protein Samples Including Aluminium Hydroxide by RP-HPLC

Aliquots (0.25 ml) of formulated OprF/I fusion protein were centrifuged at 16000×g for 10 minutes at 20° C. to separate the aluminium hydroxide sediment from the supernatant. The clear supernatant was removed and used for analysis of unbound fusion protein by RP-HPLC. The remaining pellet was resuspended in 0.25 ml of 0.1% TFA in water (pH ˜2). Samples were incubated at RT for 2 h, followed by 10 minutes centrifugation at 16.000 g at room temperature to spin down the Aluminium particles. The clear supernatant was used for analysis by RP-HPLC (TFA desorption).

### Specific Methods and Results

### Expression and Recovery of OprF/I Fusion Protein

OprF/I is a fusion protein of the pseudomonas outer membrane porin proteins OprF and OprI. It is expressed as a 224 aa fusion protein containing a His₆-tag at its N-terminus. The N-terminal Met is cleaved off after expression in E. coli. The primary structure of the expressed protein (including the N-terminal methionine) is shown in SEQ ID NO: 3.

The molecular weight of the native protein has been calculated as 24118.2 Da (full reduced protein, no N-terminal methionine). The pl has been calculated as 5.3.

The protein of the present examples is a fusion protein of outer membrane protein F and I containing a N-terminal histidine tag (His tag). The protein was expressed in E. coli XL1-Blue/pTrc-Kan-OprF/I His strain. The OprF/1-His protein was expressed intracellularly in soluble form at 30° C.

### Cell Lysis

OprF/I may be degraded by bacterial proteases, in particular when lysis buffer without high concentration of NaCl and imidazole was used. Therefore, cells were resuspended in cold lysis buffer (1:5 dilution of cell paste in buffer) consisting of 0.1 M Tris, pH 7.4, 0.5 M NaCl, 0.06 M imidazole. Addition of 0.5 M NaCl particularly inhibited proteolytic degradation of the molecule in the lysate. Resuspension and subsequent homogenization (2 cycles at 800 bar) was done at cold room temperature and the lysate was placed on ice immediately. Higher temperatures may lead to product degradation or higher protease activity.

IMAC-Copper Capture step

Chelating Sepharose FF (loaded with copper ions) was used for capturing the His-tagged

OprF/I. After loading the lysate, elution was performed with different concentrations of imidazole: 0.07 M, 0.325 M and 0.5 M imidazole. OprF/I containing fractions elute at 0.325 M imidazole as two separate peaks. Analytical data showed that RP-HPLC elution profile contained several peaks. If the same samples were analyzed under reduced conditions (addition of DTT or β-ME) only one major peak was observed. The various peaks in the untreated sample were most probably disulfide scrambled variants and aggregates of the native molecule.

An exemplary purification run was done with 992 g cell paste that is equivalent to 8.59 L of fermentation broth. After the IMAC purification and desalting on Sephadex® G50 (see below) the total amount of OprF/I was approximately 1600 mg which is equivalent to 186 mg OprF/I per liter fermentation broth.

### Desalting on Sephadex G50

This step reduced the content of low molecular weight impurities (e.g. imidazole, copper, etc.) and a buffer exchange was conducted. The loading volume was approximately 20% of the column volume. As elution buffer 0.1M Tris-HCl, 0.15M NaCl, pH 8.0 was used. It was the same buffer used for reduction and reoxidation. Alternatively, this step was also replaced by UF/DF with a 100K cut-off membrane.

### Reduction

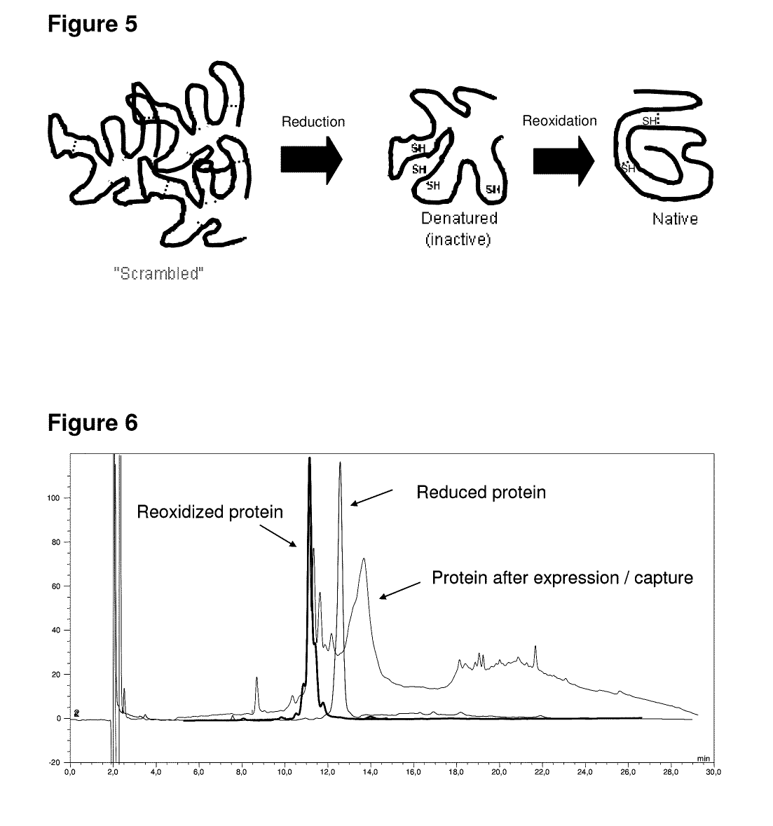

After the IMAC/G50 steps, OprF/I exists as heterogeneous mixture of misfolded forms (high and low molecular weight aggregates) caused by disulfide scrambling as schematically depicted in FIG. 5. Reduction of disulfide bonds was done with 5 mM DTT to break up all intra- and intermolecular disulfide bonds. The fully reduced protein elutes as a single peak according to RP-HPLC data. DTT can be substituted by β-ME. Since DTT is not stable over a longer period of time in aqueous solution, an aliquot of a freshly prepared DTT solution (1 M in water, used within 1 hour) is added to the IMAC/G50 pool under gentle stirring (5 mL of 1 M DTT stock solution per liter IMAC/G50 pool). The pool is incubated at room temperature for 30 minutes without stirring. Samples can be analyzed by RP-HPLC to monitor the progress of reduction.

### Reoxidation

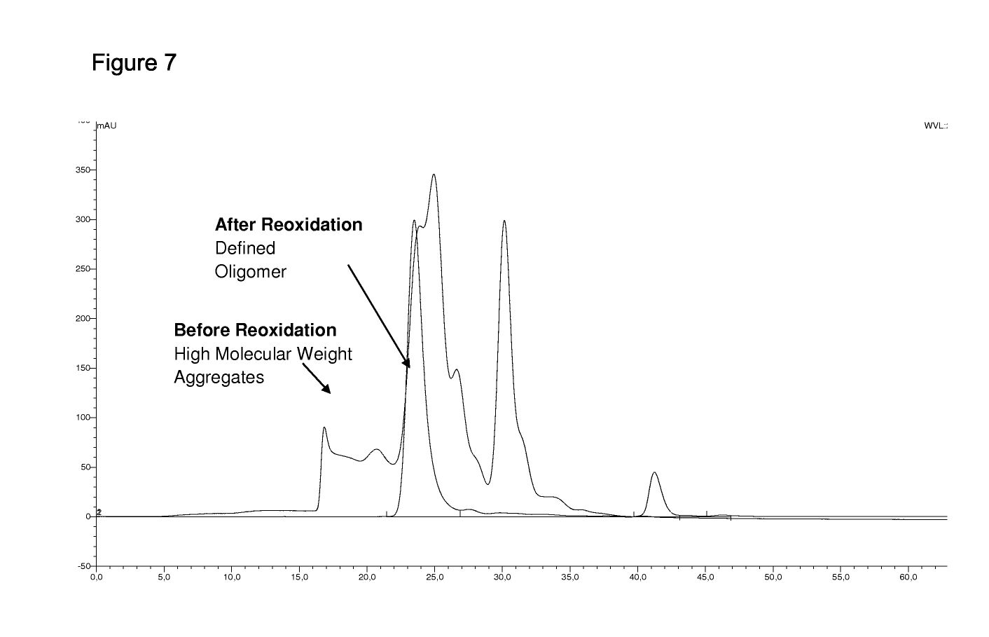

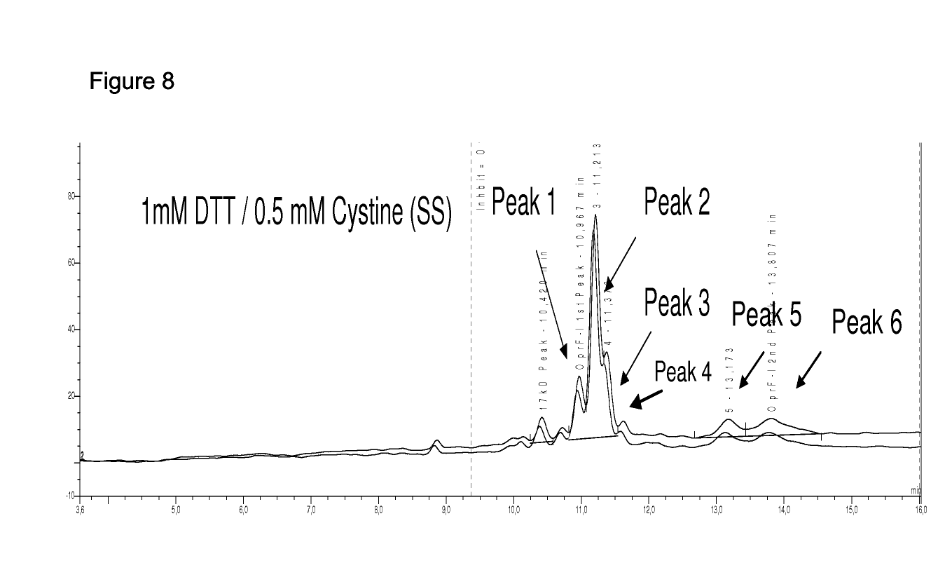

For optimization of the reoxidation conditions, different redox systems (GSSG/GSH, cystamine/cysteamine, cystine/cystein) were tested out in presence of low concentration of DTT (1 mM) to allow correct reshuffling of the disulfide bond. The progress of reoxidation (formation of disulfide bonds) can be monitored by RP-HPLC after various time intervals since the folding variants have different retention times. After preliminary studies of the various redox systems, it was decided to use cystine as the oxidizing agent. Reoxidation with cystamine/cysteamine was unsuccessful under the tested conditions. Representative RP-HPLC and SEC elution profiles prior and after reduction/reoxidation of IMAC/G50 pool are shown in FIG. 6 and FIG. 7. After reoxidation in presence of 0.5 mM cystine, the elution profiles observed by RP-HPLC and SEC were much more homogeneous compared to the “untreated” IMAC/G50 pool. The various peaks, present in the IMAC pool before reduction, shift to one major peak under reducing conditions. After reoxidation, one major peak (named as peak 2 in FIG. 8) is observed with a different retention time compared to the reduced protein. Peak 2 should represent the correctly folded OprF/I. Peak 2 is surrounded by three smaller peaks (peak 1, peak 3 and peak 4 in FIG. 8) that should be folding variants. Peaks eluting at approximately 13.17 and 13.81 min, named as peak 5 and peak 6 in FIG. 8, are other folding variants (disulfide cross-linked aggregates according to MS data).

Further characterization of peak 1 by LC-MS showed an increase in molecular weight of 240 Da compared to peak 2. This mass shift was most probably caused by covalent attachment of two molecules cystein. Free cystein was formed by the reaction of DTT with cystine, which resulted in 2 molecules cystein. It was further discovered that peak 1 increases while peak 2 decreases at increasing concentration of oxidizing agent (GSSG or cystine).

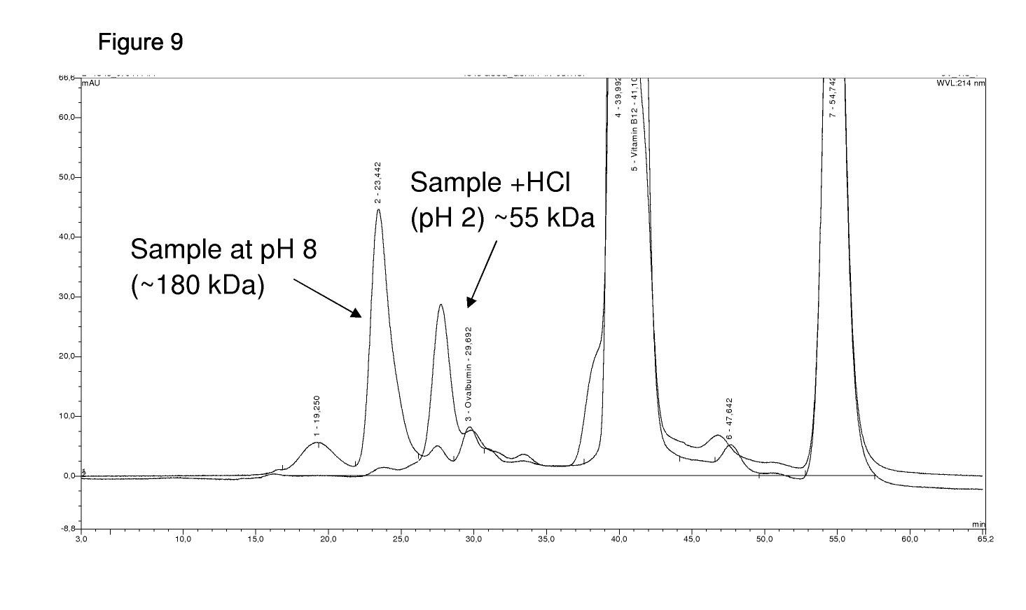

Evaluation of the main peak after reoxidation by SEC shows that the protein does not exist as a monomer. The SEC column was calibrated with reference proteins (BioRad's size exclusion standard) ranging from 1.35 to 670 kDa. The retention time of the main peak (˜25 min) corresponds to a calculated theoretical mass of ˜180 kDa under the assumption of a globular shape and no unspecific interactions with the stationary phase. It was observed that this defined multimeric state was formed preferential under the process and formulation conditions applied and seemed to be stable in aqueous solution at neutral pH in presence of NaCl. At pH 7 to 8 the OprF/I fusion protein elutes as a multimer corresponding to 180 kDa, whereas in the acidified sample (pH ˜2) the peak shifts to higher retention time (˜28 min) corresponding to approximately 55 kDa (see FIG. 9). This change in retention time could be caused by dissociation of the multimer at low pH.

In a first set of experiments, GSSG and GSH were tested out as reoxidation agents. The reduced IMAC/G50 pool in 5 mM DTT was diluted 5-fold into 0.1 M Tris-HCl, 0.15 M NaCl pH 8.0 containing GSSG (0-4 mM) under gentle stirring. DTT reacts with GSSG and forms GSH, GSSG and reduced/oxidized DTT. The final reoxidation conditions tested out covered a broad range of different ratios of GSH, GSSG and DTT. Aliquots of the samples were also quenched with HCl after various time intervals and analyzed by RP-HPLC. At increasing GSSG concentration peak 1 increases and peak 2 decreases. Formation of peak 1 occurs very early in the reoxidation process and remains constant over time. The total recovery for peaks 1+2 was estimated to be −60% starting from the completely reduced protein (100%), the recovery of all detected peaks was approximately 90% compared to the starting material.

In a second set of experiments, cystine and cystein were tested out as reoxidation agents. The reduced IMAC/G50 pool (5 mM DTT) was diluted 5-fold into 0.1 M Tris-HCl, 0.15 M NaCl pH 8.0 containing various concentration of cystine (0-3 mM) and cystein (0-3 mM). The final DTT concentration was 1 mM. Please note that the 0.2 M stock solution of cystine was prepared in 0.5 M NaOH. Samples were analyzed after 300 min and over night incubation at room temperature. No difference in RP-HPLC peak pattern for each individual experiment between the two time points was observed except for the sample containing 1 mM DTT and no cystine. The protein was still reduced after 5 h, after over night incubation peak 2 appeared. Depending on the final cystine and cystein concentration, different ratios of peak 1 and peak 2 were detected. RP-HPLC profiles showed that peak 1 concentration was lowest in presence of 0.5 mM cystine.

### Purification by DEAE Sepharose FF

Additional purification of the OprF/I containing process stream by anion exchange chromatography after reoxidation was tested out to reduce the content of remaining endotoxines and gDNA. These remaining impurities would bind to anion exchange media at neutral to slightly basic pH even at higher conductivity, whereas the product should remain in the flow through. DEAE Sepharose was tested out and found to have good properties to remove endotoxins without any major product losses by binding of OprF/I onto the resin.

### Purification by Q-Sepharose HP (QSHP)

After reoxidation and DEAE flow through chromatography, the protein solution was further purified by Q-Sepharose HP. Purification by QSHP resulted in an endotoxin concentration of ˜2 EU/mg in the main pool, which was within an acceptable low level.

### Ultrafiltration/Diafiltration

Finally, the QS-HP pool was diafiltrated against formulation buffer (1×PBS buffer pH 7.4, Dulbecco, without Ca, Mg). A 10 kDa or 30 kDa regenerated cellulose membrane (Amicon Ultra 15 centrifugal filter device, Millipore), was used. OprF/I was detected in the permeate of the 30 kDa membrane. Therefore, a 10 kDa membrane was used for final UF/DF into formulation buffer resulting in a step yield of >95%. The pool was adjusted to a final protein concentration of 1 mg/ml based on UV measurement.

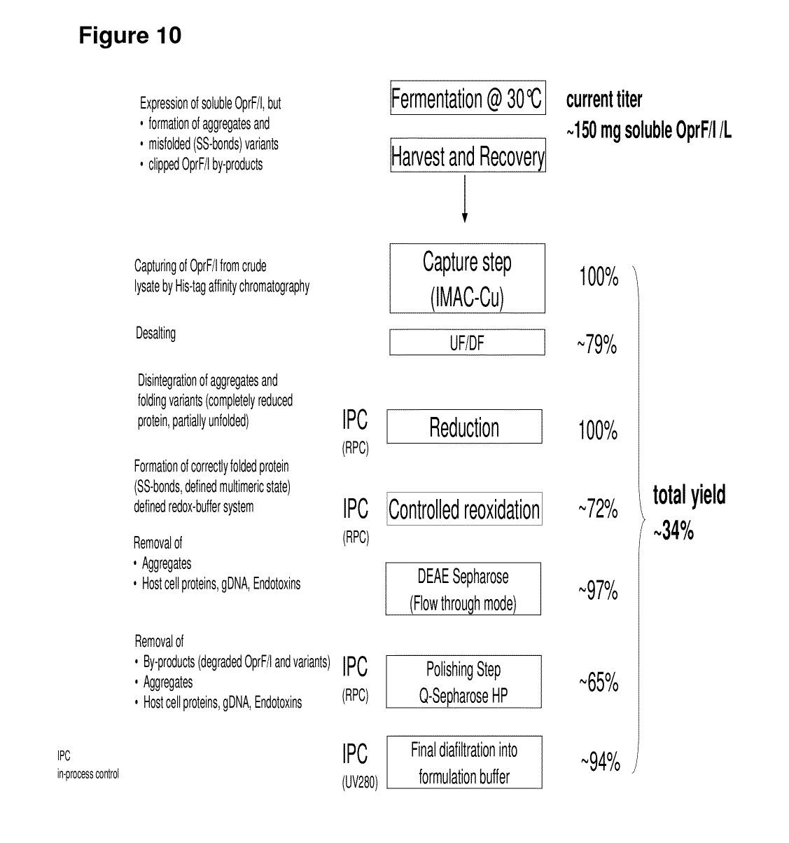

An overview of the whole production and purification process is shown in FIG. 10. An overall yield of about 34% to about 40% of purified OprF/I fusion protein was achieved.

### Characterization of the Purified OprF/I Fusion Protein

### Preparative Isolation of OprF/I Fusion Protein Variants

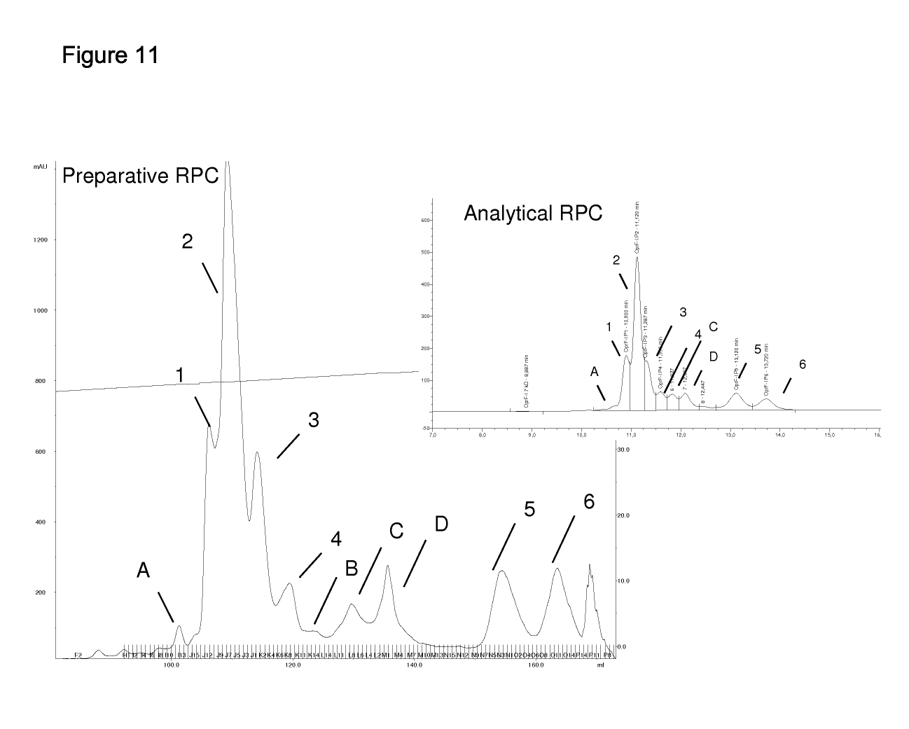

Selected side fractions from QSHP chromatography steps were used for preparative isolation. A typical preparative elution profile and nomination of peaks detected is shown in FIG. 11. All combined fractions containing the individual peaks were analyzed by SDS-PAGE and Western blot under reducing and non-reducing conditions. Under reducing conditions all bands had similar migration properties compared to an OprF/I standard. Under non-reducing conditions, the content of multimeric OprF/I variants detected at approximately 60 kDa (calibrated against the molecular weight marker) increased for Peak C, D, 5 and 6. All bands were also detected by western blot analysis using monoclonal anti OprF/I antibodies. These results indicate that all peaks detected by RP-HPLC are product related. This finding was also confirmed by peptide-mass fingerprint analysis of the individual fractions. In final DS only P1, 2, 3, 4 and 5 can be detected by RPC. The other peaks, A, B, C, D and 6, could be separated by preparative chromatography on Q-Sepharose HP from the main fractions. During Q-Sepharose HP chromatography a small peak eluted before the main peak. This fraction contained a higher concentration of an OprF/I degradation product (denoted as 7 kDa peak) as detected by analytical RP-HPLC and MALDI-ToF. This peak was also shown to be a product related fragment consisting of a 15.5 kDa and 7.2 kDa OprF/I fragment.

### Analytical Characterization of OprF/I Fusion Protein Variants

The purified OprF/I fusion protein consists of different forms of the molecule as shown by RP-HPLC (see FIG. 8). Five peaks could be detected by RP-HPLC. Peak 2 (P2) was the most prominent peak with a relative content of 50 to 55%, surrounded by peak 1 (P1), peak 3 (P3) and Peak 4 (P4). Peak 5 (P5) was well separated from the other peaks eluting at a slightly higher retention time. The relative peak content is summarized in Table 2. After reduction of the sample with β-ME or DTT, the elution profile changes. One major peak elutes and the individual variants exhibit the same chromatographic retention time. Based on these results P1 to P4 are regarded as folding variants caused by differences in disulphide bonding.

TABLE 2

| Peak | Sample 1 | Sample 2 | Sample 3 | Sample 4 |
|---|---|---|---|---|
| 1 | 19% | 14% | 13% | 11% |
| 2 | 50% | 55% | 54% | 60% |
| 3 | 18% | 17% | 19% | 14% |
| 4 | 9% | 9% | 9% | 9% |
| Sum of Peaks | 87% | 86% | 86% | 85% |
| 1, 2 and 3 | | | | |

Note:
Reoxidation of sample 1 was done in presence of 0.5 mM cystine; samples 2, 3 and 4 were reoxidized in presence of 0.375 mM cystine. The slightly higher cystine concentration resulted in minor increase in peak 1 content for sample 1.

### MALDI-ToF Analysis

For MALDI-ToF analysis the system was calibrated externally against BSA. For internal calibration Myoglobin was used. All four samples showed similar mass spectra. The main signal was from native OprF/I monomer followed by OprF/I dimer and trimer peaks. Table 3 summarizes molecular mass obtained after internal calibration. Deviation from the expected molecular mass was within the experimental error (±0.3%). Mass peaks at 24 kDa, 48 kDa and 72 kDa were detected, showing the presence of the monomeric, dimeric and trimeric OprF/I fusion proteins.

TABLE 3

| | | Deviation from |
|---|---|---|
| | | theoretical mass (Da)* |
| Peak | Analyzed mass (Da) | (rel. % deviation from theoretical MW) |
| Monomer | | |
| Sample 1 | 24096 | −20 (−0.08) |
| Sample 2 | 24053 | −63 (−0.26) |
| Sample 3 | 24097 | −19 (−0.08) |
| Sample 4 | 24045 | −71 (−0.30) |
| Dimer | | |
| Sample 1 | 48408 | +176 (+0.36) |
| Sample 2 | 48104 | −128 (−0.27) |
| Sample 3 | 48239 | −7 (−0.01) |
| Sample 4 | 48031 | −201 (−0.42) |
| Trimer | | |
| Sample 1 | 72379 | +31 (+0.04) |
| Sample 2 | 72105 | −243 (−0.34) |
| Sample 3 | 72135 | −213 (−0.30) |
| Sample 4 | 72250 | −98 (−0.14) |

*theoretical mass: monomer 24116 Da under the assumption of two disulfide bonds, dimer 48232, trimer 72348

### Native PAGE

Native PAGE of OprF/I fusion protein samples under non-reducing and reducing conditions were carried out as explained above. Band intensities after Coomassie blue staining were evaluated by densitometry. Under native conditions one OprF/I main band was detected in the range of approximately 180 kDa with a relative intensity of approximately 94 to 97%. Under reducing conditions the apparent molecular size was determined as 206 kDa. The apparent molecular weight is in good correlation with SEC-HPLC data, but different from SEC-MALS and AUC results where OprF/I mass was in the range of 80 kDa (trimer). The separation mechanism for native PAGE is the same as for native SEC, separation properties strongly depend on the shape of the protein complex when it passes through the gel. This result confirms that OprF/I has a rather elongated shape with a high hydrodynamic radius.

### N-Terminal Sequencing

The first 13 or 15 aa of two different samples were analyzed. No differences between the theoretical and detected amino acid sequence were found. The sequencing results confirmed that the N-terminal Met was completely cleaved off during expression.

### Alkylation of Thiolgroups

The results of the alkylation of the thiogroups of an OprF/I fusion protein sample showed a mass increase after alkylation of +226 Da corresponding to 4 attached molecules of iodacetamide (theoretical mass increase +228 Da; mass increase of +57 Da per attached iodacetamide molecule). This result was expected since the reduced protein contains 4 free cystein residues. All other samples did not show an increase in mass. Based on these results peak P1 of the RP-HPLC (FIG. 8) could be considered as a twofold cysteinylated variant containing one additional disulphide bond. Peaks P2 and P3 were considered as variants containing two disulphide bonds.

### Static Light Scattering (SEC/MALS)

SEC with refractive index/UV detection at 280 nm was combined with light scattering for protein characterization and molecular weight detection. As the molar mass was constant over the cross section of the main peak eluting between 23 to 26 min, a defined monodisperse molecule species eluted. For the main peak a molecular mass in the range of approx. 80 to 86 kDa was detected. The cumulative mass fraction was in the range of 94 to 98% (species 1).

The high molecular weight fraction (species 2) eluting between 20 to 22 min showed a molecular mass in the range of 140 to 190 kDa. Due to the low Rayleigh signal intensity for high molecular weight fraction the molecular mass determined exhibited a higher degree of variation. The cumulative mass fraction of species 2 was in the range of 0.5 to 1% at a range between 120 to 200 kDa.

These results exhibit that OprF/I exists as a trimer (species 1) and that only a small portion of the protein forms aggregates of higher molecular mass (species 2).

The results obtained by SEC-MALS are also in good correlation with AUC results (see below). Results obtained by SEC/UV detection and native PAGE indicated higher molecular masses for the OprF/I fusion protein in the range of 180 kDa. Results obtained by SEC and native PAGE are based on the assumption of a globular protein shape, whereas the protein shape does not influence static light scattering or AUC data. Based on the results from the different methods that were applied, it was concluded that the OprF/I trimer does not exist in a globular shape but exhibits a large hydrodynamic radius.

### Analytical Ultracentrifugation (AUC)

Sedimentation velocity profiles were recorded and deconvoluted with SedFit software to yield the sedimentation coefficient values of the sample components. The resulting calculated sedimentation coefficient and molecular mass for the individual species 1 (OprF/I fusion protein main peak) and species 2 (aggregates) were determined. The sedimentation coefficient values for the dominant component species 1 agree rather well for all samples studied. This indicates that no significant differences exist between the different samples examined. The molar mass of the main component species 1 differs within experimental variation for this parameter. It generally indicates a trimer of the OprF/I fusion protein. The molar masses of the monomer and trimer, as calculated from the sequence, are 24.1 kg/mole and 72.3 kg/mole, respectively.

No dissociation of this trimer occurred over the concentration range examined. The Stokes-radius for the trimer was calculated to be 5.6 nm. The Stokes-radius for a globular protein of the expected trimer mass is 2.8 nm. This indicates a highly asymmetrical and/or hydrated molecule. Species 2 appeared as a distinct peak at varying sedimentation coefficients. This indicates that species 2 corresponds to a component with a distinct stoichiometry (hexamer, nonamer, etc.), as opposed to unspecific aggregation. These data are in very good correlation to the SEC-MALS results showing that the native OprF/I fusion protein exists as a trimer, but are significantly different from the calculated molecular mass obtained by SEC and native PAGE (overestimation of mass due to non-globular shape). The primary and most reliable parameter from a sedimentation velocity experiment is the sedimentation coefficient itself. For the calculation of the SCD, a single frictional coefficient was assumed to apply for all sedimentation coefficients calculated. It was optimized in a fitting step. The frictional coefficient is necessary for the transformation of the SCD to a molar mass distribution (MMD). In the present study the signal for sedimentation coefficients <2 S only appeared at a ten-fold dilution. The possibility can be ruled that this peak corresponds to a putative monomer of OprF/I out because species 1 did not change. In conclusion, OprF/I is present in solution as a trimeric molecule. No dissociation occurred over the range of concentrations examined.

### Disulfide Mapping

### Disulphide Bond Mapping Using MALDI-MS/MS Analysis

The potential cysteinylation of sample peak 1 (P1) could be shown in the linear mode spectra of the tryptic digests. The peptide containing the cystein residues showed a difference of about 240 Da pointing to a cysteinylation effect (2 cysteins).

A potentially disulphide-crosslinked peptide between cystein 33 and cystein 47 of the reference sample showing a MH+ of 2100.0 Da was fragmented by MALDI-MS/MS. The two labeled cysteins are crosslinked by a disulphide bridge. This peptide was also found in samples peak 1 (P1) and peak 2 (P2) but not in sample peak 3 (P3).

### Disulphide Bond Mapping Using Nano-MS/MS Analysis

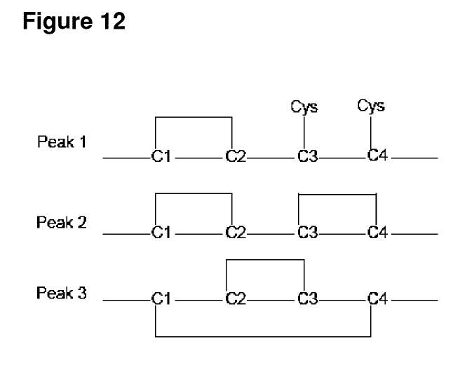

The aim of this study was to identify the differences in the disulphide bridge pattern between peaks 1, 2 and 3. The peaks were isolated and enriched. The primary sequence contains 4 cystein residues at position 18 (C1), 27 (C2), 33 (C3) and 47 (C4) (see SEQ ID NO: 1). It was concluded from the data of the intact molecular weight determination by on-line LC/ES-MS that peak 1 has one disulphide bridge and two cysteinylations, and peaks 2 and 3 have two disulphide bridges. The tryptic digest of all three peaks produced the peptide fragment 1 to 55, which contains all four cysteins of the protein. The observed masses for this fragment in the three peaks confirmed the assignment form the intact MW analysis. The peptide fragment 1 to 55 from all three peaks were collected and subdigested with AspN and analysed by LC-MS. Based on the interpretation of the raw data the structures according to FIG. 12 were derived for the predominant component in the three different peaks.

These findings were confirmed by reduction and MS/MS experiments of selected signals from the AspN subdigest. In addition to the disulphide bridge pattern deamidation was observed in the three different peaks. In the tryptic peptide 120 to 132, the Asn 124 is probably partly deamidated. In different peptides, deamidation of Asn 45 was observed as well.

### Influence of Temperature on Stability

SDS-PAGE gels (reducing and non-reducing conditions) were run for OprF/I fusion protein samples incubated at different temperatures over 10 days. Relative content of OprF/I fusion protein main band in reduced gels was calculated by densitometric evaluation of the gels by normalization of band intensities to 2-8° C. samples (reference). No degradation or changes in band pattern were observed for samples stored at −80° C., −20° C., 2-8° C. and RT (20° C.) over the storage period of 10 days.

### Influence of pH on Stability

OprF/I fusion protein samples were incubated at different pH values at pH 1.98 to pH 11.1 and analyzed by RP-HPLC and SEC-HPLC. The main peak of the OprF/I fusion protein, which corresponds to the non-covalent trimer, was constant with approximately 90% at pH 5.9 to 11.1 over the storage period of 23 days at 2-8° C. The trimer reversibly dissociated at low pH (pH 2).

### Aluminium Hydroxide as Additive/Adiuvant

RP-HPLC results showed that the OprF/I fusion protein could further be stabilized at pH 4.88 by binding onto aluminium hydroxide and could be desorbed at high recoveries.

### Immunogenicity of Different OprF/I Fusion Protein Fractions

Five BALB/c mice per group received 1 ml of different OprF/I fusion protein fractions (peaks 1, 2 and 3 of RP-HPLC fractions) and of the unfractionated OprF/I fusion protein (DS) i.p. at days 0 and 14. At day 21 the blood of the mice was tested for specific antibodies and the values (GMT [μg/ml]+SD) determined at specific doses (μg protein). The results are summarized in Table 4.

TABLE 4

| dose | Peak 1 | Peak 2 | Peak 3 | DS |
|---|---|---|---|---|
| 31.6 | 29.36 | 40.75 | 49.53 | 83.54 |
| 10 | 15.58 | 4.59 | 24.63 | 31.04 |
| 3.16 | 0.09 | 0.03 | 0.24 | 0.70 |
| 1 | 0.01 | 0.01 | 0.01 | 0.05 |
| 0.316 | 0.01 | 0.01 | 0.01 | 0.01 |

It was concluded that all fractions as well as the unfractionated OprF/I fusion protein induced specific antibodies. The ED50 value for the peak 2 fraction has additionally been determined as 5.6 μg (unfractionated OprF/I fusion protein: 1.8 μg).

### CONCLUSIONS

1. The OprF/I fusion protein and the trimeric complex thereof can be produced and purified without cross-linked disulfide aggregates in an over all yield up to 40% starting with the IMAC-Cu capture step (i.e. SEQ ID NO: 1 in the form of a trimer wherein trimer content of more than about 90% according to SEC and an aggregate content of less than 1%).
2. The OprF/I fusion protein (SEQ ID NO: 1) produced in different production lots is very consistent.
3. The OprF/I fusion protein (SEQ ID NO: 1) exists as a trimer under physiological conditions as the native outer membrane protein OprF with a mean molecular mass of approximately 80 kDa and a relative content of 94 to 98%.
4. The OprF/I fusion protein (SEQ ID NO: 1) produced according to the present invention can be separated in several variants by RP-HPLC (see FIGS. 8 and 12). Peak 1 (P1) is a two-fold cysteinylated adduct at position 33 (C3) and 47 (C4) containing a disulphide bond between position 18 (C1) and 27 (C2) (see also SEQ ID NO: 11). Peak 2 (P2) is a variant containing two disulphide bridges at positions 18 (C1) −27 (C2) and 33 (C3)-47 (C4) (see also SEQ ID NO: 12). Peak 3 (P3) is a further variant containing 2 disulphide bridges at positions 18 (C1)-47 (C4) and 27 (C2)-33 (C3) (see also SEQ ID NO: 13).
5. The OprF/I fusion protein (SEQ ID NO: 1) is stable from −80° C. to +20° C. over a period of 10 days, and at pH 5.9 to 11.1 over a period of 23 days at 2-8° C. At pH 4.88 the OprF/I fusion protein can be further stabilized by binding onto aluminium hydroxide.
6. All three variants (peaks 1, 2 and 3) as well as the unfractionated OprF/I fusion protein induced specific antibodies after vaccination of BALB/c mice.

The drug products used in the clinical trial are the Pseudomonas aeruginosa vaccines (also referred to in this experimental part as “OprF/I Vaccine”) to be injected intramuscularly that consists of a) the Ala-(His)6-OprF 190-342-OprI 21-83 protein (SEQ ID NO: 1), b) sodium chloride, c) water for injections and d) with/without aluminium hydroxide (see Table 5).

TABLE 5
Final concentrations of the drug products:

| | Concentration (Nominal Value) | |
|---|---|---|
| | in the final formulation | |
| | OprF/I Vaccine | OprF/I |
| | with Al(OH)₃, | Vaccine |
| | also referred to herein as | without |
| | alum-adjuvanted vaccine | Al(OH)₃ |
| Drug substance: Ala-(His)6- | 100 mcg/ml | 100 mcg/ml |
| OprF 190-342-Oprl 21-83 | | |
| protein (SEQ ID NO: 1) - | | |
| protein in PBS buffer | | |
| Sodium chloride 0.9% (NaCl) | 0.81%¹ | 0.81%¹ |
| Aluminium hydroxide Al(OH)₃ | 400 mcg/ml | — |

¹Calculated theoretical value: not considering any salt components already present in the drug substance

Production of drug products: The frozen drug substance (Ala-(His)6-OprF 190-342-OprI 21-83 protein (SEQ ID NO: 1)-protein in PBS buffer) is thawed overnight at 2-8° C. and formulated with 0.9% sodium chloride solution to reach the above sodium final concentration. The final formulation is sterile filtered right before filling. For the Alum adjuvanted drug product sterile Al(OH)₃ is added after the sterile filtration step. The 1 ml dose aliquots of 1.2 ml (extractable volume 1 ml) are aseptically filled into sterile pyrogen-free glass vials.

The placebo consists of a commercially available and registered physiological NaCl Product (NaCl 0.9%, Isotone sodium chloride 0.9% Braun; 5 mL Mini-Plasco® connect). It is provided in 5 ml containers and registered for intravenous and subcutaneous application. It is stored at ambient temperature. To fully mimic the vaccines the placebo consists of PBS diluted tenfold with 0.9% Saline, with 400 mcg/ml Al(OH)₃ added. Its nominal volume is 1 ml, filled in 2 ml glass vials. It should be stored at 2-8° C. The placebo for phase 2 clinical trial has been formulated and filled at the same manufacturers, using analogous processes, as the drug product with aluminium hydroxide.

### B. Clinical Trial

### Randomized, Placebo-Controlled, Partially Blinded Phase 2 Pilot Study Design:

400 male or female patients admitted to an ICU with need for mechanical ventilation for more than 48 hours, aged between 18 and 80 years were vaccinated on days zero and seven in four treatment groups receiving 100 or 200 mcg alum-adjuvanted OprF/I Vaccine (SEQ ID NO: 1), 100 mcg non-adjuvanted OprF/I Vaccine (SEQ ID NO: 1) or alum as placebo control (see above material section above for a more detailed description of the tested drug products). Study duration per patient was estimated to be 90 days and overall study duration was estimated to be 12 to 18 months.

The following endpoints were measured:

### Primary:

Immunogenicity at day 14 as determined by OprF/I specific IgG antibody titer measured by ELISA in patients receiving OprF/I Vaccine (SEQ ID NO: 1) or placebo

### Secondary:

Immunogenicity at day 7 and in biweekly intervals after day 14 until hospital discharge, day of ICU discharge and at day 90 as determined by OprF/I specific IgG antibody titer measured by ELISA in patients receiving OprF/I vaccine or placebo
Rate of serious adverse events and adverse events during the vaccination period up to 90 days after the first vaccination
Safety laboratory parameters (hematology, serum chemistry, urinalysis) up to 90 days after the first vaccination
Systemic tolerability (Vital signs: blood pressure, pulse, body temperature)
Local tolerability (local injection site reactions)
Number of patients with invasive infection with P. aeruginosa, such as bacteremia (determined as positive blood culture) or P. aeruginosa pneumonia (determined according to NNIS VAP criteria) in patients receiving OprF/I vaccine or placebo up to day 90
Number of patients with P. aeruginosa tracheobronchitis, P. aeruginosa positive wounds, urine and respiratory secretion cultures in patients receiving OprF/I vaccine or placebo
Overall survival of patients receiving OprF/I vaccine or placebo
Length of stay in ICU and hospital
Time to onset of VAP in patients receiving OprF/I vaccine or placebo
Antibiotic-free days in patients receiving OprF/I vaccine or placebo
Prevalence of infections due to pathogens others than P. aeruginosa in patients receiving OprF/I vaccine or placebo
Organ function (Sequential Organ Failure Assessment [SOFA] scores)
Presence of anti-histidine antibodies in patients receiving OprF/I vaccine or placebo at day 7, 14, 90 and day of ICU discharge
Measurement of functional IgG antibodies by OPA at day 7, 14, in biweekly intervals until hospital discharge, day of ICU discharge, day 90
Measurement of avidity of OprF/I specific IgG antibodies at day 7, 14, day of ICU discharge and day 90

### Results of the Study:

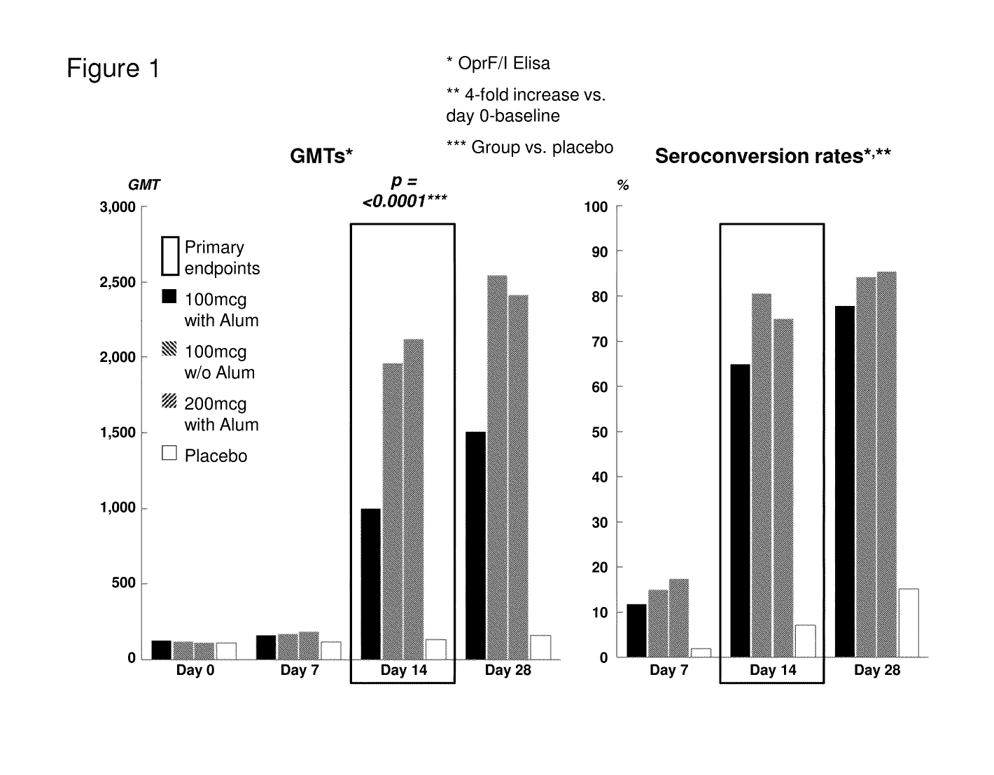

The primary endpoint of the study was met in that all vaccine groups showed good seroconversion (i.e., at least 4-fold increase in OprF/I IgG until Day 14 after first vaccination) rates (65 to 81% in OprF/I Vaccine (SEQ ID NO: 1) treatment arms) with IgG antibody Geometric Mean Titers (GMTs) that were significantly higher in all OprF/I Vaccine (SEQ ID NO: 1) treatment arm compared to placebo group (GMTs in OprF/I Vaccine (SEQ ID NO: 1) groups: 995-2117 ELISA units/ml) at day 14 (FIG. 1). This is an approximately 4 fold increase in OprF/I IgG from day 0 to day 14. There were no significant differences in treatment emergent adverse events between the treatment arms and local and systemic tolerability appeared to be good, as far as assessable in this study population. The number and nature of reported drug related adverse events does not raise any safety concern and was confirmed by a Data Safety Monitoring Board (DSMB) based on interim data.

Secondary immunogenicity endpoints were also met in this study and included IgG response assessed seven times over a period of 90 days, and included measurement of functional antibody activity by opsonophagocytosis assay, and measurement of antibody avidity. Overall robust immunogenicity following second vaccination was observed in all vaccine groups. A dose response could be observed, whereas the non-alum adjuvanted vaccine was at least as immunogenic as the alum-adjuvanted vaccine. Antibody avidity was similar in all vaccine groups. Functional opsonization uptake could be shown and correlated well with vaccine-induced IgG titers. Immune responses in intensive care patients appeared weaker compared to results from a preceding Phase I trial in healthy volunteers. This was not unexpected due to the reduced general health condition of patients enrolled.

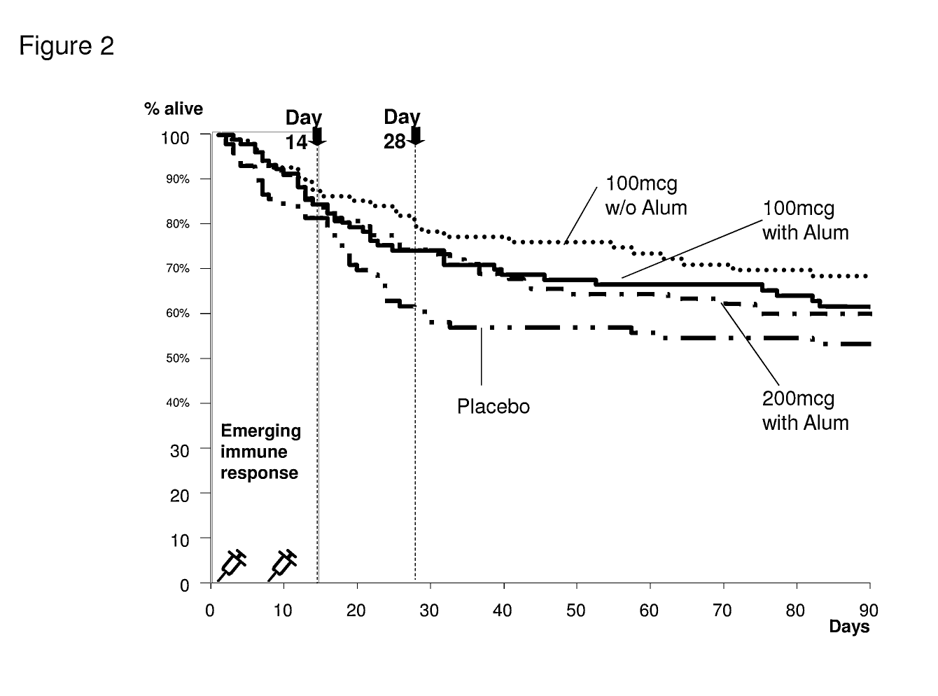

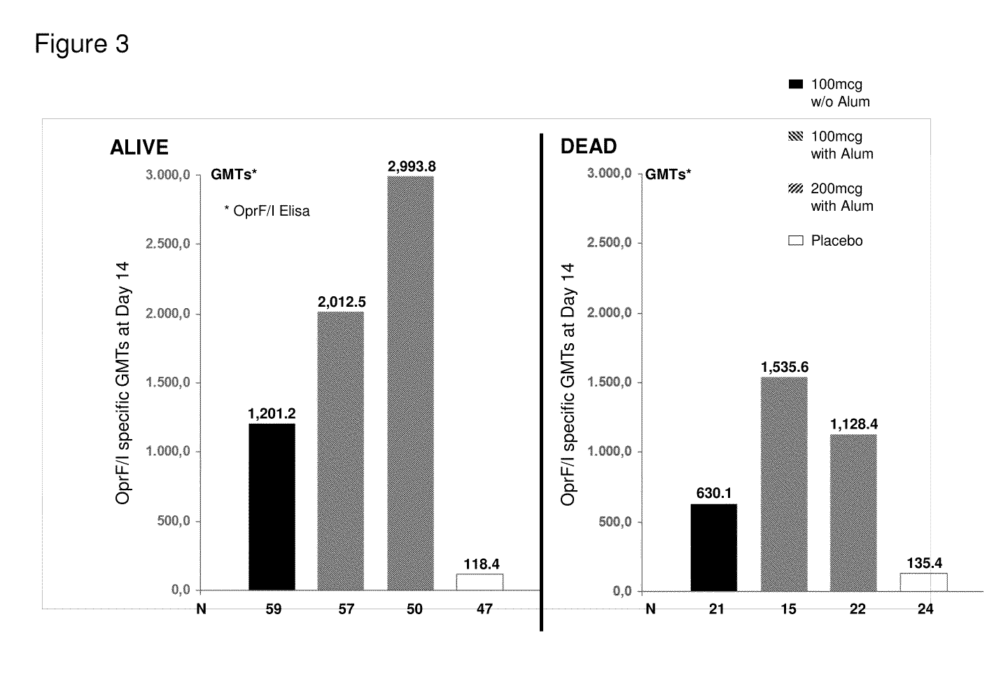

Although this trial was not powered for efficacy the Clinical Endpoint Committee (CEC) confirmed infection rates and mortality were recorded within the secondary endpoints analysis. A lower mortality rate was observed in all vaccine groups as compared to the control group (FIG. 2). The reduction in mortality rate was statistically significant (p=0.0196) for the non-adjuvanted vaccine (21.7% day 28 mortality in the not-adjuvanted OprF/I vaccine group compared to 40.0% day 28 mortality in the placebo group). Patients who survived until study end had higher OprF/I IgG titers at day 14 after first vaccination compared to patients who died beyond day 14 (FIG. 3). Cox regression analysis demonstrated a significant prognostic value of the OprF/I IgG titer on survival (p=0.0336).

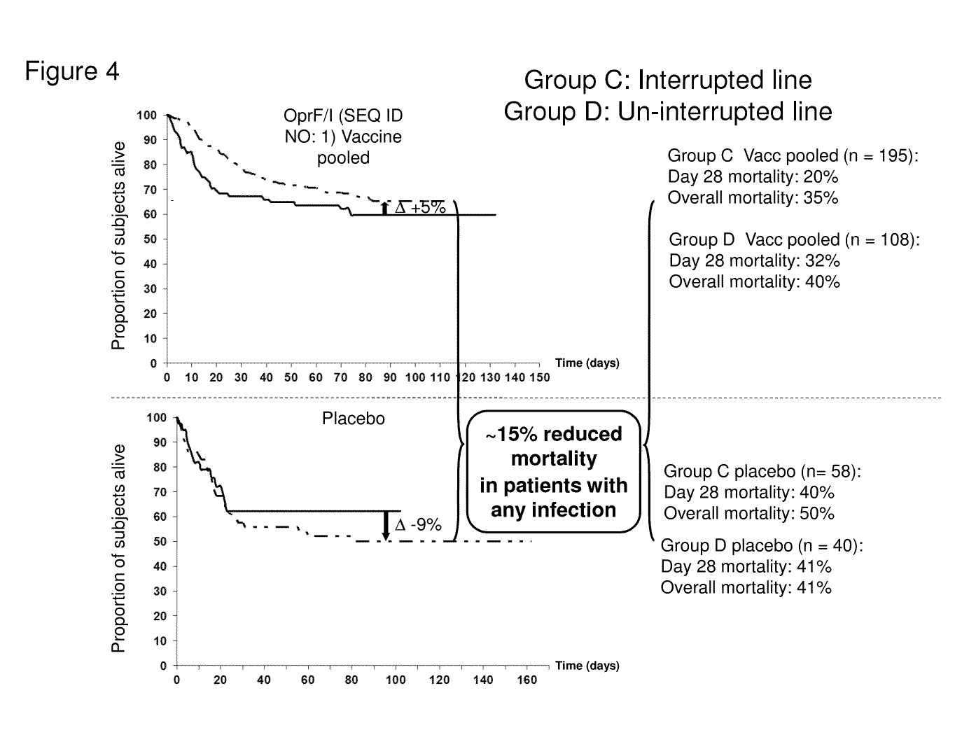

No significant difference in Pseudomonas aeruginosa infection rates between any of the groups was apparent. However, this may be attributed to the relatively small sample size of the current Phase II study and the methodological limitations of reliable Pseudomonas aeruginosa diagnosis. Moreover, vaccination with OprF/I Vaccine (SEQ ID NO: 1) might influence virulence rather than clearance of P. aeruginosa infection including the course of other infections. The latter assumption is supported by the observation that OprF/I Vaccine (SEQ ID NO: 1) vaccinated patients having experienced any kind of infection (regardless of pathogen) during the course of the study showed an approximately 15% lower mortality compared to patients with any kind of infection (due to any pathogen) but vaccinated with placebo (FIG. 4).

Regarding the observed approximately 15% reduction of mortality in patients with any kind of infection treated with non alum adjuvanted OprF/I Vaccine (SEQ ID NO: 1) versus alum adjuvanted placebo, it can only be speculated about the underlying mechanism. The vaccine might reduce virulence of Pseudomonas aeruginosa rather than providing sterilizing immunity. OprF (part of the vaccine antigen) can bind human Interferon-gamma thereby altering the expression of virulence factors of P. aeruginosa (Wu L. et al. Recognition of host immune activation by Pseudomonas aeruginosa. Science, 2005; 309: 774-7). Antibodies against OprF induced by OprF/I vaccine block this interaction (Bin Ding et al. Vaccine 2010; 28:4119-22).

Reducing virulence of P. aeruginosa may indirectly reduce the frequency of subsequent infections with other pathogens. In this regard it is important to highlight the notorious difficulty in diagnosis of P. aeruginosa, which limits establishing causal relationship between P. aeruginosa infection and mortality. Finally, immunization in general has an immunomodulatory effect that may positively influence the course of the infections.

Larger, sufficiently powered clinical studies would be required to validate and verify any vaccine effects on mortality and infection rates.

As another key objective, the current Phase II trial investigated the feasibility of performing pivotal efficacy studies in this difficult target population: Final data confirm the anticipated number of Pseudomonas aeruginosa infections. The observed attack rate of 6 to 14% is well within expectations as only study sites with estimated Pseudomonas aeruginosa invasive infection rates of 10 to 25% were selected for this trial.

### Preferred Aspects

1. A method of reducing mortality in a human such as e.g. a hospitalized patient comprising administering to said patient a pharmaceutically effective amount of an OprF/I agent.
2. A method of reducing mortality in an ICU patient comprising administering to said patient a pharmaceutically effective amount of an OprF/I agent.
3. A method of reducing mortality in a ventilated ICU patient comprising administering to said patient a pharmaceutically effective amount of an OprF/I agent.
4. A method of reducing mortality in a burn victim comprising administering to said victim a pharmaceutically effective amount of an OprF/I agent.
5. A method of reducing mortality in a cystic fibrosis patient comprising administering to said patient a pharmaceutically effective amount of an OprF/I agent.
6. A method according to any one of aspects 1 to 5, comprising co-administration of a therapeutically effective amount of an OprF/I agent and a second drug substance, said second drug substance being an antimicrobial or antifungal drug.
7. A method according to any one of aspects 1 to 5, wherein the administration of the therapeutically effective amount of an OprF/I agent is given twice at an amount of 100 mcg.
8. A method according to any one of aspects 2 to 3, wherein the administration of the therapeutically effective amount of an OprF/I agent is given at least 2 weeks before admission to the ICU.
9. A method according to any one of the preceding aspects, wherein the OprF/I agent is a compound selected from the group consisting of polypeptides with SEQ ID NOs: 1 to 13, the trimeric forms thereof, and antibodies direct against to any of said polypeptides.
10. A method according to any one of the preceding aspects, wherein the OprF/I agent is selected from the group consisting of SEQ ID NO: 11, SEQ ID NO: 12 and SEQ ID NO: 13, the trimeric forms thereof including trimeric forms with mixtures of SEQ ID NO: 11, SEQ ID NO: 12 and SEQ ID NO: 13, and antibodies direct against any of said polypeptides or trimeric forms.
11. An OprF/I agent for use in any method according to any one of the preceding aspects.
12. An OprF/I agent for use in the reduction of mortality of a hospitalized patient, an ICU patient, a cystic fibrosis patient, a ventilated ICU patient, or a burn victim.
13. An OprF/I agent for use in the reduction of mortality according to aspect 12, wherein the OprF/I agent is a polypeptide with SEQ ID NO: 1.
14. A pharmaceutical composition for use in any method according to any one of the aspects 1 to 10, comprising an OprF/I agent together with one or more pharmaceutically acceptable diluents or carriers therefor.
15. A pharmaceutical combination comprising:
   a) a first agent which is an OprF/I agent, and
   b) a co-agent which is an antimicrobial or antifungal drug.
16. The method of any of aspects 1 to 10, the OprF/I agent of any of aspects 11 to 13 or the pharmaceutical composition of aspects 14 or 15, wherein the OprF/I agent is a fusion protein comprising or consisting of the Pseudomonas aeruginosa outer membrane protein I fused with its amino-terminal end to the carboxy-terminal end of a carboxy-terminal portion of the Pseudomonas aeruginosa outer membrane protein F, particularly
   (i) wherein the Pseudomonas aeruginosa outer membrane protein I is the full length outer membrane protein I;
   (ii) wherein the Pseudomonas aeruginosa outer membrane protein F is the full length outer membrane protein F;
   (iii) wherein the OprF/I fusion protein consists of or comprises SEQ ID NO: 1;
   (iv) wherein the OprF/I fusion protein consists of or comprises antibody or fragment thereof directed against said polypeptide or SEQ ID NO: 1;
   (v) wherein the OprF/I fusion protein is a functional active variant and/or has at least 50% sequence identity to SEQ ID NO: 1, especially at least 60%, preferably at least 70%, more preferably at least 80%, still more preferably at least 90%, even more preferably at least 95%, most preferably 99% sequence identity;
   (vi) wherein the fusion protein forms a trimer comprising (or consisting of at least 80%, 85%, 90% or 95% of) a polypeptide with SEQ ID NO: 1 or immunogenic variants thereof with 80%, 85%, 90%, or 95% identity to SEQ ID NO: 1;
   (vii) wherein the fusion protein forms a trimer comprising (or consisting of at least 80%, 85%, 90% or 95% of) a polypeptide with SEQ ID NO: 1 or immunogenic variants thereof with 80%, 85%, 90%, or 95% identity to SEQ ID NO: 1 and wherein said polypeptide or variant thereof have either a) a Cys18-Cys27-bond (see e.g. SEQ ID NO: 11), b) a Cys18-Cys27-bond and a Cys33-Cys47-bond (see e.g. SEQ ID NO: 12), or c) Cys18-Cys47-bond and Cys27-Cys33-bond (see e.g. SEQ ID NO: 13).
17. A method for producing the OprF/I fusion protein according to any above aspects, said method comprising the steps of
   (a) reducing said OprF/I fusion protein with a reducing agent, and
   (b) oxidizing the reduced OprF/I fusion protein with a redox agent, in the presence of a reducing agent.
18. The method according to aspect 17, wherein in step (a) the concentration of the reducing agent is from about 3 mM to about 10 mM and the reducing agent is dithiothreitol (DTT), dithioerythritol (DTE) or β-mercaptoethanol.
19. The method according to aspects 17 or 18, wherein in step (b) the concentration of the redox agent is from about 0.2 mM to about 4 mM and the redox agent is glutathione disulfide/glutathione or the redox agent cystine/cysteine, and the concentration of the reducing agent is from about 0.375 mM to about 1.5 mM and the reducing agent is dithiothreitol (DTT), dithioerythritol (DTE) or β-mercaptoethanol.
20. The method according to any of the aspects 17 to 19, wherein the reaction temperature is from about 18° C. to about 25° C.

1. A method of reducing mortality in a subject, comprising administering to said subject a therapeutically effective amount of an OprF/I agent, wherein said subject is selected from the group consisting of:
i) a hospitalized patient or a subject who is at risk of being hospitalized;
ii) an ICU patient or a subject who is at risk of being admitted to ICU;
iii) a ventilated ICU patient or a subject who is at of risk being ventilated;
iv) a burn victim patient; and
v) a cystic fibrosis patient.

2. The method of claim 1, comprising co-administration of a therapeutically effective amount of an OprF/I agent and a second drug substance, wherein said second drug substance is an antimicrobial or antifungal drug.

3. The method of claim 1, wherein the OprF/I agent is administered twice to the subject at an amount of 100 mcg.

4. The method of claim 1, wherein the OprF/I agent is administered to the subject at least 2 weeks before admission to the hospital.

5. The method of claim 1, wherein the OprF/I agent comprises a polypeptide selected from the group consisting of polypeptides with SEQ ID NOs: 1 to 13.

6. The method of claim 1, wherein the OprF/I agent comprises a polypeptide a polypeptide of SEQ ID NO: 1.

7. The method of claim 1, wherein the OprF/I agent comprises a protein complex, wherein the protein complex is a trimer of OprF/I fusion proteins of SEQ ID NO: 1 or functional active variants thereof having at least 85% identity to the amino acid sequence of SEQ ID NO: 1.

8. The method of claim 7, wherein at least 80% of the OprF/I agent consists of the protein complex according to claim 7.

9. The method of claim 7, wherein the OprF/I fusion protein is selected from the group consisting of
(a) the OprF/I fusion protein of SEQ ID NO: 1 with a Cys18-Cys27-bond (SEQ ID NO: 11), and
(b) the OprF/I fusion protein of SEQ ID NO: 1 with a Cys18-Cys27-bond and Cys33-Cys47-bond (SEQ ID NO: 12), and
(c) the OprF/I fusion protein of SEQ ID NO: 1 with a Cys18-Cys47-bond and Cys27-Cys33-bond (SEQ ID NO: 13),
or functional active variants thereof having at least 85% identity to the amino acid sequence of SEQ ID NO: 1 and the same disulfide bond pattern as specified in (a), (b) or (c).

10. The method of claim 9, wherein the sum of a) the OprF/I fusion protein of SEQ ID NO: 1 with a Cys18-Cys27-bond (SEQ ID NO: 11), b) the OprF/I fusion protein of SEQ ID NO: 1 with a Cys18-Cys27-bond and Cys33-Cys47-bond (SEQ ID NO: 12), and c) the OprF/I fusion protein of SEQ ID NO: 1 with a Cys18-Cys47-bond and Cys27-Cys33-bond (SEQ ID NO: 13) is equal or greater than 75% of the total OprF/I fusion protein of SEQ ID NO: 1.

11. The method of claim 7, wherein the OprF/I agent is the OprF/I fusion protein of SEQ ID NO: 1 with a Cys18-Cys27-bond (SEQ ID NO: 11) or a functional active variant thereof having at least 85% identity to the amino acid sequence of SEQ ID NO: 1, and the same disulfide bond pattern as specified in SEQ ID NO: 11.

12. The method of claim 7, wherein the OprF/I agent is the OprF/I fusion protein of SEQ ID NO: 1 with a Cys18-Cys27-bond and Cys33-Cys47-bond (SEQ ID NO: 12) or a functional active variant thereof having at least 85% identity to the amino acid sequence of SEQ ID NO: 1, and the same disulfide bond pattern as specified in SEQ ID NO: 12.

13. The method of claim 7, wherein the OprF/I agent is the OprF/I fusion protein of SEQ ID NO: 1 with a Cys18-Cys47-bond and Cys27-Cys33-bond (SEQ ID NO: 13) or a functional active variant thereof having at least 85% identity to the amino acid sequence of SEQ ID NO: 1, and the same disulfide bond pattern as specified in SEQ ID NO: 13.

14. The method of claim 1, wherein the OprF/I agent is a hybrid protein comprising the Pseudomonas aeruginosa outer membrane protein I fused with its amino-terminal end to the carboxy-terminal end of a carboxy-terminal portion of the Pseudomonas aeruginosa outer membrane protein F, particularly
(i) wherein the Pseudomonas aeruginosa outer membrane protein I is the full length outer membrane protein I; especially SEQ ID NO: 5;
(ii) wherein the Pseudomonas aeruginosa outer membrane protein F is the full length outer membrane protein F, especially SEQ ID NO: 6;
(iii) wherein the OprF/I agent consists of or comprises SEQ ID NO: 1;
(iv) wherein the OprF/I agent is a functional active variant and/or has at least 85% sequence identity to SEQ ID NO: 1;
(v) wherein the OprF/I agent forms a trimer comprising or consisting of a polypeptide with SEQ ID NO: 1 or functional active variants thereof with at least 85% identity to SEQ ID NO: 1;
(vi) wherein the OprF/I agent forms a trimer comprising or consisting of a polypeptide of SEQ ID NO: 1 with (a) a Cys18-Cys27-bond (see e.g. SEQ ID NO: 11), or (b) a Cys18-Cys27-bond and a Cys33-Cys47-bond (see e.g. SEQ ID NO: 12), or (c) Cys18-Cys47-bond and Cys27-Cys33-bond (see e.g. SEQ ID NO: 13), or (d) functional active variants with at least 85% identity to SEQ ID NO: 1 and the same disulfide bond pattern as specified in (a), (b) or (c), or e) combinations of (a), (b), (c) and/or (d);
(vii) wherein the OprF/I agent comprises or consist of i) SEQ ID NO: 2, 3, 7 to 10, and/or ii) SEQ ID NO: 4;
(viii) wherein the OprF/I agent comprises the carboxy terminal portion of outer membrane protein F with the sequence from amino acid 190 to amino acid 342 of the native OprF protein (SEQ ID NO: 3) or the carboxy terminal portion of outer membrane protein F with the sequence from amino acid 190 to amino acid 350 of the native OprF protein (SEQ ID NO: 2);
(ix) wherein the OprF/I agent comprises the amino-terminal end of outer membrane protein I with the sequence from amino acid 21 to amino acid 83 of the native OprI protein (SEQ ID NO: 4);
(x) wherein the OprF/I agent comprises or consists of the carboxy terminal portion of outer membrane protein F with the sequence from amino acid 190 to amino acid 342 of the native OprF protein (SEQ ID NO: 3) or the carboxy terminal portion of outer membrane protein F with the sequence from amino acid 190 to amino acid 350 of the native OprF protein (SEQ ID NO: 2) fused to the amino-terminal end of outer membrane protein I with the sequence from amino acid 21 to amino acid 83 of the native OprI protein (SEQ ID NO: 4); and/or
(xi) wherein the OprF/I agent comprises an Ala-(His)₆ tag such as e.g. in the OprF/I agent of SEQ ID NO: 1.

16. The method of claim 1, wherein the OprF/I agent comprises optionally at least one additive or adjuvant.

17. The method of claim 16, wherein the adjuvant is aluminium hydroxide, preferably formulated in an isotonic phosphate buffer (pH 7.4).

18. A method of reducing mortality in a subject comprising
administering to said subject a therapeutically effective amount of an antibody or antigen-binding portions thereof specifically binding the OprF/I agent, wherein said subject is selected from the group consisting of:
i) a hospitalized patient or a subject who is at risk of being hospitalized;
ii) an ICU patient or a subject who is at risk of being admitted to ICU;
iii) a ventilated ICU patient or a subject who is at of risk being ventilated;
iv) a burn victim patient; and
v) a cystic fibrosis patient.

19. The method of claim 18, wherein the OprF/I agent comprises a polypeptide selected from the group consisting of polypeptides with SEQ ID NOs: 1 to 13.

20. The method of claim 18, wherein the OprF/I agent comprises polypeptide a polypeptide of SEQ ID NO: 1.

21. The method of claim 18, wherein the OprF/I agent is selected from the group consisting of
(a) the OprF/I fusion protein of SEQ ID NO: 1 with a Cys18-Cys27-bond (SEQ ID NO: 11), and
(b) the OprF/I fusion protein of SEQ ID NO: 1 with a Cys18-Cys27-bond and Cys33-Cys47-bond (SEQ ID NO: 12), and
(c) the OprF/I fusion protein of SEQ ID NO: 1 with a Cys18-Cys47-bond and Cys27-Cys33-bond (SEQ ID NO: 13),
or a functional active variants thereof having at least 85% identity to the amino acid sequence of SEQ ID NO: 1 and the same disulfide bond pattern as specified in (a), (b) or (c).

22. The method of claim 18, wherein the OprF/I agent is a hybrid protein comprising the Pseudomonas aeruginosa outer membrane protein I fused with its amino-terminal end to the carboxy-terminal end of a carboxy-terminal portion of the Pseudomonas aeruginosa outer membrane protein F, particularly
(i) wherein the Pseudomonas aeruginosa outer membrane protein I is the full length outer membrane protein I; especially SEQ ID NO: 5;
(ii) wherein the Pseudomonas aeruginosa outer membrane protein F is the full length outer membrane protein F, especially SEQ ID NO: 6;
(iii) wherein the OprF/I agent consists of or comprises SEQ ID NO: 1;
(iv) wherein the OprF/I agent is a functional active variant and/or has at least 85% sequence identity to SEQ ID NO: 1;
(v) wherein the OprF/I agent forms a trimer comprising or consisting of a polypeptide with SEQ ID NO: 1 or functional active variants thereof with at least 85% identity to SEQ ID NO: 1;
(vi) wherein the OprF/I agent forms a trimer comprising or consisting of a polypeptide of SEQ ID NO: 1 with (a) a Cys18-Cys27-bond (see e.g. SEQ ID NO: 11), or (b) a Cys18-Cys27-bond and a Cys33-Cys47-bond (see e.g. SEQ ID NO: 12), or (c) Cys18-Cys47-bond and Cys27-Cys33-bond (see e.g. SEQ ID NO: 13), or (d) functional active variants with at least 85% identity to SEQ ID NO: 1 and the same disulfide bond pattern as specified in (a), (b) or (c), or e) combinations of (a), (b), (c) and/or (d);
(vii) wherein the OprF/I agent comprises or consist of i) SEQ ID NO: 2, 3, 7 to 10, and/or ii) SEQ ID NO: 4;
(viii) wherein the OprF/I agent comprises the carboxy terminal portion of outer membrane protein F with the sequence from amino acid 190 to amino acid 342 of the native OprF protein (SEQ ID NO: 3) or the carboxy terminal portion of outer membrane protein F with the sequence from amino acid 190 to amino acid 350 of the native OprF protein (SEQ ID NO: 2);
(ix) wherein the OprF/I agent comprises the amino-terminal end of outer membrane protein I with the sequence from amino acid 21 to amino acid 83 of the native OprI protein (SEQ ID NO: 4); and/or
(x) wherein the OprF/I agent comprises or consists of the carboxy terminal portion of outer membrane protein F with the sequence from amino acid 190 to amino acid 342 of the native OprF protein (SEQ ID NO: 3) or the carboxy terminal portion of outer membrane protein F with the sequence from amino acid 190 to amino acid 350 of the native OprF protein (SEQ ID NO: 2) fused to the amino-terminal end of outer membrane protein I with the sequence from amino acid 21 to amino acid 83 of the native OprI protein (SEQ ID NO: 4).


SEQUENCE LISTING (13 sequences)

SEQ ID NO: 1 - protein, 223 aa, Artificial Sequence
  Ala-(His)6-OprF190-342-OprI21-83
AHHHHHHAPAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVESSHSKETEARLTATEDAAARAQARADEAYRKADEALGAAQKAQQTADEANERALRMLEKASRK

SEQ ID NO: 2 - protein, 161 aa, Artificial Sequence
  OprF190-350
APAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEAEVEAEAK

SEQ ID NO: 3 - protein, 153 aa, Artificial Sequence
  OprF190-342
APAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVE

SEQ ID NO: 4 - protein, 63 aa, Artificial Sequence
  OprI21-83
SSHSKETEARLTATEDAAARAQARADEAYRKADEALGAAQKAQQTADEANERALRMLEKASRK

SEQ ID NO: 5 - protein, 83 aa, Pseudomonas aeruginosa
MNNVLKFSALALAAVLATGCSSHSKETEARLTATEDAAARAQARADEAYRKADEALGAAQKAQQTADEANERALRMLEKASRK

SEQ ID NO: 6 - protein, 350 aa, Pseudomonas aeruginosa
MKLKNTLGVVIGSLVAASAMNAFAQGQNSVEIEAFGKRYFTDSVRNMKNADLYGGSIGYFLTDDVELALSYGEYHDVRGTYETGNKKVHGNLTSLDAIYHFGTPGVGLRPYVSAGLAHQNITNINSDSQGRQQMTMANIGAGLKYYFTENFFAKASLDGQYGLEKRDNGHQGEWMAGLGVGFNFGGSKAAPAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEAEVEAEAK

SEQ ID NO: 7 - protein, 30 aa, Artificial Sequence
  OprF212-240
NVDKCPDTPANVTVDANGCPAVAEVVRVQL

SEQ ID NO: 8 - protein, 14 aa, Artificial Sequence
  OprF243-256
KFDFDKSKVKENSY

SEQ ID NO: 9 - protein, 14 aa, Artificial Sequence
  OprF285-298
TDAYNQKLSERRAN

SEQ ID NO: 10 - protein, 18 aa, Artificial Sequence
  OprF332-350
EGRAINRRVEAEVEAEAK

SEQ ID NO: 11 - protein, 223 aa, Artificial Sequence; identical to SEQ ID NO: 1
  Ala-(His)6-OprF190-342-OprI21-83, disulfide bond on residues 18-27

SEQ ID NO: 12 - protein, 223 aa, Artificial Sequence; identical to SEQ ID NO: 1
  Ala-(His)6-OprF190-342-OprI21-83, disulfide bonds on residues 18-27 and 33-47

SEQ ID NO: 13 - protein, 223 aa, Artificial Sequence; identical to SEQ ID NO: 1
  Ala-(His)6-OprF190-342-OprI21-83, disulfide bonds on residues 18-47 and 27-33